(19) United States
(12) Patent Application Publication
(10) Pub. No.: US 2022/0195535 A1
(43) Pub. Date: Jun. 23, 2022

(54) Methods for Detecting and Treating Esophageal Cancer

(71) Applicant: The Johns Hopkins University, Baltimore, MD (US)

(72) Inventors: Stephen J. Meltzer, Lutherville, MD (US); Ke Ma, Hanover, MD (US); Yulan Cheng, Ellicott City, MD (US); John Martin Abraham, Woodbine, MD (US)

(21) Appl. No.: 17/604,255

(22) PCT Filed: Apr. 16, 2020
(86) PCT No.: PCT/US2020/028459

§ 371 Date: Oct. 15, 2021

Related U.S. Application Data
(60) Provisional application No. 62/834,447, filed on Apr. 16, 2019.

(51) Int. Cl. C12Q 1/6886

(52) U.S. Cl. CPC C12Q 1/6886; C12Q 2600/154

(57) ABSTRACT

The present invention relates to the field of cancer. More specifically, the present invention provides compositions

and methods useful for detecting and treating esophageal cancer. In a specific embodiment, a method for identifying a subject having esophageal squamous cell carcinoma (ESCC) comprises (a) extracting genomic DNA from a sample obtained from the subject; (b)

performing a conversion reaction on the genomic DNA in vitro to convert unmethylated cytosine to uracil by deamination; and (c)

detecting nucleic acid methylation of one or more genes in the converted genomic DNA, wherein detecting nucleic acid methylation e identifies the subject as having ESCC. The one or more genes can comprise ZNF542, ZNF132, cg20655070, TAC1 and SLC35F1. In a

more specific embodiment, the one or more genes comprise ZNF542 and ZNF132 and can further comprise detecting the nucleic acid N methylation of one or more of cg20655070, TAC1 and SLC35F1.

### CROSS-REFERENCE TO RELATED APPLICATIONS

This application claims the benefit of U.S. Provisional Application No. 62/834,447, filed Apr. 16, 2019, which is incorporated herein by reference in its entirety.

### STATEMENT OF GOVERNMENTAL INTEREST

This invention was made with government support under grant no. CA211457, awarded by the National Institutes of Health. The government has certain rights in the invention.

### FIELD OF THE INVENTION

The present invention relates to the field of cancer. More specifically, the present invention provides compositions and methods useful for detecting and treating esophageal cancer.

### INCORPORATION-BY-REFERENCE OF MATERIAL SUBMITTED ELECTRONICALLY

This application contains a sequence listing. It has been submitted electronically via EFS-Web as an ASCII text file entitled “P15702-02_ST25.txt.” The sequence listing is 21,167 bytes in size, and was created on Apr. 15, 2020. It is hereby incorporated by reference in its entirety.

### BACKGROUND OF THE INVENTION

Esophageal cancer is the 7th-most common cancer and 6^{th}-most common cause of cancer-related deaths worldwide, with 572,000 new cases and 509,000 deaths in 2018 (International Agency for Research on Cancer (IARC), Globocan 2018). The form of esophageal cancer that predominates in developed nations is esophageal adenocarcinoma (EAC). In contrast, esophageal squamous cell carcinoma (ESCC) accounts for over 80% of all esophageal cancer deaths globally, and is greatly predominant in low- and middle-income countries (LMICs). ESCC incidence rates vary widely, and geographic areas with disproportionately high incidence rates exist in eastern Africa, northern China, northeastern Iran and southern South America (1). Within numerous countries in eastern Africa, ESCC ranks as the third-most common cancer and is a leading cause of death.

Reflecting the significant impact of ESCC in the developing world, the NCI and IARC convened an International Tumor Workshop on Esophageal Squamous Cell Carcinoma in September 2016 (1). While this meeting was attended by international experts representing multiple high-incidence regions throughout the developing world, a substantial focus was on the emerging data and unmet needs due to the high burden of this disease in East Africa. One outcome of this meeting was the establishment of the African Esophageal Cancer Consortium (2).

Currently, the gold standard method for diagnosis of ESCC involves the use of esophagogastroduodenoscopic (EGD) equipment. The limited availability of endoscopy facilities, among the myriad healthcare challenges faced by patients in LMICs, has particularly negative consequences for patients at high risk for this deadly disease. Unfortunately, very few ESCC patients in LMICs in East Africa have access to endoscopy facilities, and thus endoscopic screening for ESCC is not feasible. As a result, patients in these settings typically present with advanced disease with its accompanying high morbidity and mortality rates.

### SUMMARY OF THE INVENTION

In response to the need for a low-cost and widely available assay for screening and early detection of ESCC in high-risk populations, the present inventors have developed an assay that utilizes DNA methylation biomarkers and, in some embodiments, a non-invasive sample retrieval sponge. In certain embodiments, the present invention identifies ESCC in asymptomatic patients and thereby selects patients who should be prioritized for endoscopic evaluation.

In one aspect, the present invention provides methods for identifying a subject having esophageal squamous cell carcinoma (ESCC). In one embodiment, the method comprises (a) extracting genomic DNA from a sample obtained from the subject; (b) performing a conversion reaction on the genomic DNA in vitro to convert unmethylated cytosine to uracil by deamination; and (c) detecting nucleic acid methylation of one or more genes in the converted genomic DNA, wherein detecting nucleic acid methylation identifies the subject as having ESCC. In a specific embodiment, the one or more genes comprise ZNF542, ZNF132, cg20655070, TAC1 and SLC35F1. The present invention contemplates using at least one, at least two, at least three, at least four or all five of the recited genes. In a more specific embodiment, the one or more genes comprise ZNF542 and ZNF132 and, in a further embodiment, step (c) further comprises detecting the nucleic acid methylation of one or more of cg20655070, TAC1 and SLC35F1.

In certain embodiments, the detecting step (c) comprises a polymerase chain reaction (PCR)-based technique. In a specific embodiment, the PCR-based technique is quantitative methylation specific PCR (QMSP). Exemplary primers/probes useful in such methods are described SEQ ID NOS. 1-15 (see Table 1 below).

In particular embodiments, steps (a) and (b) are performed using methylation on beads technique. In certain embodiments, the sample is a cell sample. In specific embodiments, the cell sample is retrieved using a swallowable sponge device. The method can further comprise a step (d) of performing an endoscopy on the subject.

In particular embodiments, the conversion reaction on the ZNF542 gene results in the sequence shown in nucleotides 1-578 of SEQ ID NO:17. In other embodiments, the qMSP reaction utilizes primers that amplify the sequence shown in nucleotides 115-230 of SEQ ID NO:17. In a specific embodiment, a kit comprises such primers.

In certain embodiments, the conversion reaction on the ZNF132 gene results in the sequence shown in nucleotides 1-1036 of SEQ ID NO:19. In specific embodiments, the qMSP reaction utilizes primers that amplify the sequence shown in nucleotides 285-420 of SEQ ID NO:19. In a specific embodiment, a kit comprises such primers.

In particular embodiments, the conversion reaction on the cg20655070 gene results in the sequence shown in nucleotides 1-618 of SEQ ID NO:21. In other embodiments, the qMSP reaction utilizes primers that amplify the sequence shown in nucleotides 204-324 of SEQ ID NO:21. In a specific embodiment, a kit comprises such primers.

In certain embodiments, the conversion reaction on the TACT gene results in the sequence shown in nucleotides 1-1886 of SEQ ID NO:23. In specific embodiments, the qMSP reaction utilizes primers that amplify the sequence shown in nucleotides 103-208 of SEQ ID NO:23. In a specific embodiment, a kit comprises such primers.

In particular embodiments, the conversion reaction on the SLC35F1 gene results in the sequence shown in nucleotides 1-1769 of SEQ ID NO:25. In other embodiments, the qMSP reaction utilizes primers that amplify the sequence shown in nucleotides 867-1014 of SEQ ID NO:25. In a specific embodiment, a kit comprises such primers.

In another aspect, the present invention provides methods for treating a subject having ESCC. In one embodiment, the methods comprises the steps of: (a) extracting genomic DNA from a sample obtained from the subject; (b) performing a conversion reaction on the genomic DNA in vitro to convert unmethylated cytosine to uracil by deamination; (c) detecting nucleic acid methylation of one or more genes in the converted genomic DNA, wherein detecting nucleic acid methylation identifies the subject as having ESCC; and (d) administering to the subject one or more treatment modalities appropriate for a subject having ESCC.

In particular embodiments, the one or more treatment modalities comprises endoscopic resection, surgery, chemotherapy, radiotherapy or combinations thereof. Further and more specific treatment modalities are described herein. In a specific embodiment, an endoscopy is performed prior to the treatment of step (d). The one or more genes can comprise ZNF542, ZNF132, cg20655070, TAC1 and SLC35F1. In a specific embodiment, the one or more genes comprise ZNF542 and ZNF132 and, in another embodiment, step (c) further comprises detecting the nucleic acid methylation of one or more of cg20655070, TAC1 and SLC35F1. In certain embodiments, the detecting step (c) comprises a polymerase chain reaction (PCR)-based technique. In a specific embodiment, the PCR-based technique is quantitative methylation specific PCR (QMSP). In particular embodiments, steps (a) and (b) are performed using methylation on beads technique. In certain embodiments, the sample is a cell sample. In specific embodiments, the cell sample is retrieved using a swallowable sponge device.

In certain embodiments, a method comprises the steps of: (a) extracting genomic DNA from a sample obtained from the subject; (b) performing a conversion reaction on the genomic DNA in vitro to convert unmethylated cytosine to uracil by deamination; and (c) detecting nucleic acid methylation of ZNF542 and ZNF132 in the converted genomic DNA. In another embodiment, step (c) further comprises detecting nucleic acid methylation of one or more of cg20655070, TAC1 and SLC35F1. In certain embodiments, the detecting step (c) comprises a polymerase chain reaction (PCR)-based technique. In a specific embodiment, the PCR-based technique is quantitative methylation specific PCR (QMSP). In particular embodiments, steps (a) and (b) are performed using methylation on beads technique. In certain embodiments, the sample is a cell sample. In specific embodiments, the cell sample is retrieved using a swallowable sponge device. The method can further comprise a step (d) of performing an endoscopy on the subject.

In another aspect, the present invention provides kits. In one embodiment, a kit comprises: (a) a primer complementary to a bisulfite-converted nucleic acid sequence comprising a CpG dinucleotide in the ZNF542 gene; and (b) a primer complementary to a bisulfite-converted nucleic acid sequence comprising a CpG dinucleotide in the ZNF132 gene. In a specific embodiment, (a) comprises one or more of SEQ ID NOS:1-3. In one embodiment, (b) comprises one or more of SEQ ID NOS:4-6.

The kit can further comprise one or more of: (c) a primer complementary to a bisulfite-converted nucleic acid sequence comprising a CpG dinucleotide in the cg20655070 gene; (d) a primer complementary to a bisulfite-converted nucleic acid sequence comprising a CpG dinucleotide in the TAC1 gene; and (e) a primer complementary to a bisulfite-converted nucleic acid sequence comprising a CpG dinucleotide in the SLC35F1 gene. In a specific embodiment, (c) comprises one or more of SEQ ID NOS:7-9. In another specific embodiment, (d) comprises one or more of SEQ ID NOS:10-12. In yet another specific embodiment, (e) comprises on or more of SEQ ID NOS:13-15.

### BRIEF DESCRIPTION OF THE FIGURES

FIG. 1. Earlier detection of ESCC using proposed diagnostic strategy. Current ESCC diagnosis relies on the availability and use of EGD. Too often, this cancer is not detected until patients present with dysphagia, obstruction and resulting malnutrition, resulting in high mortality and morbidity. Use of the EsophaCap™ sponge and EsoCAN gene methylation panel is rapid and inexpensive, and has the potential to result in cancer detection at earlier stages. In addition, the large worldwide market will support commercialization in LMICs throughout the world.

FIG. 2. The EsophaCap™ swallowable sponge device. The collapsible black plastic sponge is compressed in a soluble gelatin capsule and tethered to a filament. The filament is held outside the mouth while the capsule is swallowed. Inside the stomach, the capsule dissolves and the sponge expands. It is then retrieved by pulling on the filament. Cytologic material attaches during exit, including diseased and normal cells.

FIG. 3. Methylation levels of 5 genes in 48 matched normal and ESCC tissue samples. DNA was extracted and bisulfite-modified from biopsy tissues and analyzed using the methylation on beads (MOB) technique for methylation relative to beta-actin. These five genes demonstrated statistically significant discrimination between ESCC and normal esophageal tissues.

FIG. 4. Gene methylation status in EsophaCap™ sponge samples. Methylation levels of five genes were determined using the MOB procedure on EsophaCap™ sponge samples from 12 ESCC and 14 non-neoplastic control patients.

FIG. 5A-5B. ROC plots and classification formula for sponge samples. FIG. 5A: Each sample was obtained from an EsophaCap™ sponge, and methylation indexes were obtained for the five genes shown. Samples were obtained from 12 ESCC patients and 14 non-neoplastic controls. LASSO with logistic regression was applied to generate the two-gene model displayed in thick dark blue. FIG. 5B: two-marker model and score calculation for ESCC.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “gene” is a reference to one or more gene, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

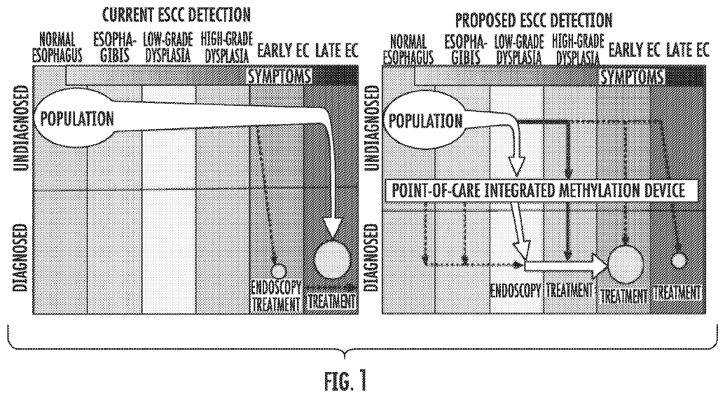

The current gold-standard for ESCC detection method, EGD, is sensitive and specific but highly expensive, risky (1-3% combined mortality and complication rate), inconvenient (causing lost productivity for patient and accompanying persons), and constrained by the availability of healthcare personnel with formal training to perform it. Thus, alternative diagnostic modalities are desperately needed in areas with a high incidence of ESCC. The present invention comprises a strategy for ESCC detection and screening based on DNA methylation biomarkers. In a particular embodiment, the present invention leverages an FDA-cleared, CE-marked swallowable retrievable sponge (EsophaCap™) to collect esophageal cells in combination with a molecular biomarker ESCC assay. The implementation of this minimally invasive, inexpensive strategy results in earlier ESCC diagnosis and decreased ESCC mortality in resource constrained settings (FIG. 1). This technology could result in a paradigm shift for this deadly cancer in populations affected by a high incidence of ESCC.

Thus, in particular embodiments, the present invention provides minimally invasive compositions and methods to detect certain markers based on direct access to neoplastic cells residing in the esophagus. In certain embodiments, esophageal cells are collected and subsequently maintained using a protocol that allows for near-indefinite storage of the samples prior to assay, providing the flexibility needed to accommodate the variety of clinical and diagnostic settings that occur in LMICs. The approach thus provides a simple, low-cost method for early ESCC detection that is specifically designed to address a major health issue in technologically disadvantaged areas of the world.

The assay relies on advances in methods to detect gene methylation in rare human cell populations. In particular embodiments, a physical platform (magnetic bead) is also used to increase the accuracy, sensitivity and reproducibility of epigenetic profiles that are obtained from scarce esophageal cells that are recovered directly from patients.

The assay allows for convenient and inexpensive sampling of organ-specific mucosa in at-risk patients in low-resource settings. EsophaCap™ samples can be collected without medical personnel, such as physicians or nurses. Furthermore, samples can be transported at room temperature in storage fluid to laboratories anywhere in the world for analysis. The ease of use of the sampling technique, coupled with the ability to perform the molecular assays in standard diagnostic laboratories, will enhance adoption of the assay by disadvantaged populations. As a result, it is expected that the assay will ultimately reduce the burden of ESCC in areas where the disease is currently most prevalent and deadly.

DNA does not exist as naked molecules in the cell. For example, DNA is associated with proteins called histones to form a complex substance known as chromatin. Chemical modifications of the DNA or the histones alter the structure of the chromatin without changing the nucleotide sequence of the DNA. Such modifications are described as “epigenetic” modifications of the DNA. Changes to the structure of the chromatin can have a profound influence on gene expression. If the chromatin is condensed, factors involved in gene expression may not have access to the DNA, and the genes will be switched off.

Conversely, if the chromatin is “open,” the genes can be switched on. Some important forms of epigenetic modification are DNA methylation and histone deacetylation. DNA methylation is a chemical modification of the DNA molecule itself and is carried out by an enzyme called DNA methyltransferase. Methylation can directly switch off gene expression by preventing transcription factors binding to promoters. A more general effect is the attraction of methyl-binding domain (MBD) proteins. These are associated with further enzymes called histone deacetylases (HDACs), which function to chemically modify histones and change chromatin structure. Chromatin-containing acetylated histones are open and accessible to transcription factors, and the genes are potentially active. Histone deacetylation causes the condensation of chromatin, making it inaccessible to transcription factors and causing the silencing of genes.

CpG islands are short stretches of DNA in which the frequency of the CpG sequence is higher than other regions. The “p” in the term CpG indicates that cysteine (“C”) and guanine (“G”) are connected by a phosphodiester bond. CpG islands are often located around promoters of housekeeping genes and many regulated genes. At these locations, the CG sequence is not methylated. By contrast, the CG sequences in inactive genes are usually methylated to suppress their expression.

About 56% of human genes and 47% of mouse genes are associated with CpG islands. Often, CpG islands overlap the promoter and extend about 1000 base pairs downstream into the transcription unit. Identification of potential CpG islands during sequence analysis helps to define the extreme 5′ ends of genes, something that is notoriously difficult with cDNA-based approaches. The methylation of a CpG island can be determined by a skilled artisan using any method suitable to determine such methylation. For example, the skilled artisan can use a bisulfite reaction-based method for determining such methylation.

The present invention provides methods to determine the nucleic acid methylation of ZNF542, ZNF132, cg20655070, TAC1 and/or SLC35F1, of a patient. In certain embodiments, the methylation status of one or more of such genes can be used to predict the clinical course and eventual outcome of patients suspected of being predisposed or of having a neoplasm such as ESCC.

In particular, in certain embodiments of the disclosure, the methods may be practiced as follows. A sample is taken from a patient. In certain embodiments, a single cell type may be isolated for further testing. The DNA is harvested from the sample and examined to determine if the region(s) of ZNF542, ZNF132, cg20655070, TAC1 and/or SLC35F1 is/are methylated. For example, the DNA of interest can be treated with bisulfite to deaminate unmethylated cytosine residues to uracil. Because uracil base pairs with adenosine, thymidines are incorporated into subsequent DNA strands in the place of unmethylated cytosine residues during subsequence PCR amplifications. Next, the target sequence is amplified by PCR, and probed with a specific probe. Only DNA from the patient that was methylated will bind to the probe.

Methods of determining the patient nucleic acid profile are well known to the art worker and include any of the well-known detection methods. Various PCR methods are described, for example, in PCR Primer: A Laboratory Manual, Dieffenbach 7 Dveksler, Eds., Cold Spring Harbor Laboratory Press, 1995. Other analysis methods include, but are not limited to, nucleic acid quantification, restriction enzyme digestion, DNA sequencing, hybridization technologies, such as Southern Blotting, etc., amplification methods such as Ligase Chain Reaction (LCR), Nucleic Acid Sequence Based Amplification (NASBA), Self-sustained Sequence Replication (SSR or 3SR), Strand Displacement Amplification (SDA), and Transcription Mediated Amplification (TMA), Quantitative PCR (qPCR), or other DNA analyses, as well as RT-PCR, in vitro translation, Northern blotting, and other RNA analyses. In another embodiment, hybridization on a microarray is used.

### I. Definitions

By “alteration” is meant an increase or decrease. An alteration may be by as little as 1%, 2%, 3%, 4%, 5%, 10%, 20%, 30%, or by 40%, 50%, 60%, or even by as much as 75%, 80%, 90%, or 100%. An alteration may be a change in sequence relative to a reference sequence or a change in expression level, activity, or epigenetic marker (e.g., promoter methylation).

By “control” is meant a standard or reference condition, For example, the methylation level present at a promoter in a neoplasia may be compared to the level of methylation present at that promoter in a corresponding normal tissue.

“Detect” refers to identifying the presence, absence or amount of the analyte to be detected.

By “clinical aggressiveness” is meant the severity of a neoplasia. Aggressive neoplasias are more likely to metastasize than less aggressive neoplasias. While conservative methods of treatment are appropriate for less aggressive neoplasias, more aggressive neoplasias may require more aggressive therapeutic regimens.

The term “agent” means a polypeptide, polynucleotide, or fragment, or analog thereof, small molecule, inhibitory RNA, or other biologically active molecule.

The term “nucleic acid” refers to deoxyribonucleotides or ribonucleotides and polymers thereof in either single- or double-stranded form, made of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues. The terms “nucleic acid,” “nucleic acid molecule,” or “polynucleotide” are used interchangeably and may also be used interchangeably with gene, cDNA, DNA and/or RNA encoded by a gene.

The term “nucleotide sequence” refers to a polymer of DNA or RNA which can be single-stranded or double-stranded, optionally containing synthetic, non-natural or altered nucleotide bases capable of incorporation into DNA or RNA polymers. A DNA molecule or polynucleotide is a polymer of deoxyribonucleotides (A, G, C, and T), and an RNA molecule or polynucleotide is a polymer of ribonucleotides (A, G, C and U).

A “gene,” for the purposes of the present invention, includes a DNA region encoding a gene product, as well as all DNA regions which regulate the production of the gene product, whether or not such regulatory sequences are adjacent to coding and/or transcribed sequences. The term “gene” is used broadly to refer to any segment of nucleic acid associated with a biological function. Genes include coding sequences and/or the regulatory sequences required for their expression. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. For example, “gene” refers to a nucleic acid fragment that expresses mRNA, functional RNA, or specific protein, including regulatory sequences. “Functional RNA” refers to sense RNA, antisense RNA, ribozyme RNA, siRNA, or other RNA that may not be translated but yet has an effect on at least one cellular process. “Genes” also include non-expressed DNA segments that, for example, form recognition sequences for other proteins. “Genes” can be obtained from a variety of sources, including cloning from a source of interest or synthesizing from known or predicted sequence information, and may include sequences designed to have desired parameters.

“Gene expression” refers to the conversion of the information, contained in a gene, into a gene product. It refers to the transcription and/or translation of an endogenous gene, heterologous gene or nucleic acid segment, or a transgene in cells. In addition, expression refers to the transcription and stable accumulation of sense (mRNA) or functional RNA Expression may also refer to the production of protein. The term “altered level of expression” refers to the level of expression in transgenic cells or organisms that differs from that of normal or untransformed cells or organisms.

A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation. The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA “Messenger RNA” (mRNA) refers to the RNA that is without intrans and that can be translated into protein by the cell. “cDNA” refers to a single- or a double-stranded DNA that is complementary to and derived from mRNA. “Functional RNA” refers to sense RNA, antisense RNA, ribozyme RNA, siRNA, or other RNA that may not be translated but yet has an effect on at least one cellular process.

A “coding sequence,” or a sequence that “encodes” a selected polypeptide, is a nucleic acid molecule that is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral (e.g., DNA viruses and retroviruses) or prokaryotic DNA, and especially synthetic DNA sequences. A transcription termination sequence may be located 3′ to the coding sequence.

Certain embodiments of the disclosure encompass isolated or substantially purified nucleic acid compositions. In the context of the present invention, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived.

By “fragment” is intended a polypeptide consisting of only a part of the intact full-length polypeptide sequence and structure. The fragment can include a C-terminal deletion an N-terminal deletion, and/or an internal deletion of the native polypeptide. A fragment of a protein will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, or any integer between 5 amino acids and the full-length sequence.

Certain embodiments of the disclosure encompass isolated or substantially purified nucleic acid compositions. In the context of the present invention, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule is substantially free of other cellular material or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived.

“Naturally occurring” is used to describe a composition that can be found in nature as distinct from being artificially produced. For example, a nucleotide sequence present in an organism, which can be isolated from a source in nature and which has not been intentionally modified by a person in the laboratory, is naturally occurring.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, intrans, and polyadenylation signal sequences. They include natural and synthetic sequences as well as sequences that may be a combination of synthetic and natural sequences.

A “5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency. A “3′ non-coding sequence” refers to nucleotide sequences located 3′ (downstream) to a coding sequence and may include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor. The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

A “promoter” refers to a nucleotide sequence, usually upstream (5′) to its coding sequence, which directs and/or controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions. “Constitutive expression” refers to expression using a constitutive promoter. “Conditional” and “regulated expression” refer to expression controlled by a regulated promoter.

“Operably-linked” refers to the association of nucleic acid sequences on a single nucleic acid fragment so that the function of one of the sequences is affected by another. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation of an endogenous gene, heterologous gene or nucleic acid segment, or a transgene in cells. In addition, expression refers to the transcription and stable accumulation of sense (mRNA) or functional RNA Expression may also refer to the production of protein. The term “altered level of expression” refers to the level of expression in cells or organisms that differs from that of normal cells or organisms.

The term “epigenetic marker” or “epigenetic change” refers to a change in the DNA sequences or gene expression by a process or processes that do not change the DNA coding sequence itself. In an exemplary embodiment, methylation is an epigenetic marker.

By “frequency of methylation” is meant the number of times a specific promoter is methylated in a number of samples.

By “increased methylation” is meant a detectable positive change in the level, frequency, or amount of methylation. Such an increase may be by 5%, 10%, 20%, 30%, or by as much as 40%, 50%, 60%, or even by as much as 75%, 80%, 90%, or 100%. In certain embodiments, the detection of any methylation in a promoter in a subject sample is sufficient to identify the subject as having a neoplasia, a pre-cancerous lesion, or the propensity to develop a neoplasia.

The term “hypermethylation” refers to the presence of methylated alleles in one or more nucleic acids. In preferred embodiments, hypermethylation is detected using methylation specific polymerase chain reaction (MSP).

As used herein, “methylation” is meant to refer to cytosine methylation at positions C5 of cytosine, the N6 position of adenine or other types of nucleic acid methylation. Methylation can be detection by, for example, by polymerase chain reaction (PCR), including, but not limited to methylation specific PCR. Portions of the DNA regions described herein will comprise at least one potential methylation site (i.e., a cytosine) and can comprise 2, 3, 4, 5, 6, 7, 8, 9, 10, or more potential methylation sites. In preferred embodiments, methylation is detected using methylation specific polymerase chain reaction (MSP).

As used herein the term “methylation status” is meant to refer to the presence, absence and/or quantity of methylation at a particular nucleotide, or nucleotides within a portion of DNA. The methylation status of a particular DNA sequence (e.g., a DNA marker or DNA region as described herein such as, for example, ZNF542, ZNF132, cg20655070, TACT and SLC35F1, and the like) may indicate the methylation state of every base in the sequence or can indicate the methylation state of a subset of the base pairs (e.g., cytosines or the methylation state of one or more specific restriction enzyme recognition sequences) within the sequence, or can indicate information regarding regional methylation density within the sequence without providing precise information of where in the sequence the methylation occurs. In certain embodiments, the methylation status can optionally be represented or indicated by a “methylation value.” A methylation value can be generated, for example, by quantifying the amount of intact DNA present following restriction digestion with a methylation dependent restriction enzyme. In this example, if a particular sequence in the DNA is quantified using quantitative PCR, an amount of template DNA approximately equal to a mock treated control indicates the sequence is not highly methylated whereas an amount of template substantially less than occurs in the mock treated sample indicates the presence of methylated DNA at the sequence. Accordingly, a value, i.e., a methylation value, for example from the above described example, represents the methylation status and can thus be used as a quantitative indicator of methylation status. This is of particular use when it is desirable to compare the methylation status of a sequence in a sample to a threshold value. In certain examples, the methylation status is determined for a particular gene such as, for example, aZNF542, ZNF132, cg20655070, TACT and SLC35F1. In preferred embodiments, methylation is detected using methylation specific polymerase chain reaction (MSP).

By “methylation level” is meant the number of methylated alleles of a particular gene. Methylation level may be represented as the methylation present at a target gene/reference gene×100. Any ratio that allows the skilled artisan to distinguish neoplastic tissue from normal tissue is useful in the methods of the invention. In various embodiments, a methylation ratio cutoff value is 1, 2, 3, 4, 5, 6, or 7. One skilled in the art appreciates that the cutoff value is selected to optimize both the sensitivity and the specificity of the assay. In certain embodiments, merely detecting promoter methylation of the genes ZNF542, ZNF132, cg20655070, TAC1 and SLC35F1 in a biological sample of a subject is sufficient to identify the subject as having cancer, a pre-cancerous lesion, or having a propensity to develop cancer.

By “tumor marker profile” is meant an alteration present in a subject sample relative to a reference. In one embodiment, a tumor marker profile includes promoter methylation of a gene such as, for example, ZNF542, ZNF132, cg20655070, TAC1 and SLC35F1, as well as other markers known in the art.

By “methylation profile” is meant the methylation level at two or more promoters. In one embodiment, promoter methylation of a gene such as, for example, ZNF542, ZNF132, cg20655070, TAC1 and SLC35F1 is detected.

By “sensitivity” is meant the percentage of subjects with a particular disease that are correctly detected as having the disease. For example, an assay that detects 98/100 of carcinomas has 98% sensitivity.

By “severity of neoplasia” is meant the degree of pathology. The severity of a neoplasia increases, for example, as the stage or grade of the neoplasia increases.

By “specificity” is meant the percentage of subjects without a particular disease who test negative.

The term “neoplasm” or “neoplasia” as used herein refers to inappropriately high levels of cell division, inappropriately low levels of apoptosis, or both. A neoplasm creates an unstructured mass (e.g., a tumor), which can be either benign or malignant. For example, cancer is a neoplasia. Examples of cancers include, without limitation, lung carcinoma, small cell lung carcinoma, non-small cell lung carcinoma, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma. In a specific embodiment, the neoplasia is ESCC.

The term “sample” as used herein refers to any biological or chemical mixture for use in the method of the invention. The sample can be a biological sample. The biological samples are generally derived from a patient, including a cell sample or bodily fluid (such as tumor tissue, lymph node, sputum, blood, bone marrow, cerebrospinal fluid, phlegm, saliva, or urine) or cell lysate. The cell lysate can be prepared from a tissue sample (e.g., a tissue sample obtained by biopsy), for example, a tissue sample (e.g., a tissue sample obtained by biopsy), blood, cerebrospinal fluid, phlegm, saliva, urine, or the sample can be cell lysate. In preferred examples, the sample is one or more of blood, blood plasma, serum, cells, a cellular extract, a cellular aspirate, tissues, a tissue sample, or a tissue biopsy. In preferred embodiments, the sample is from esophageal tumor cells, tissue or origin.

By “marker” is meant any protein or polynucleotide having an alteration in methylation, expression level or activity that is associated with a disease or disorder.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

The term “stage” or “staging” as used herein is meant to refer to the extent or progression of proliferative disease, e.g., cancer, in a subject. Staging can be “clinical” and is according to the “stage classification” corresponding to the TNM classification (Rinsho, Byori, Genpatsusei Kangan Toriatsukaikiyaku (Clinical and Pathological Codes for Handling Primary Liver Cancer): 22p. Nihon Kangangaku Kenkyukai (Liver Cancer Study Group of Japan) edition (3rd revised edition), Kanehara Shuppan, 1992). Staging in certain embodiments may refer to “molecular staging” as defined by nucleic acid hypermethylation of one or more genes in one or more samples. In preferred embodiments of the invention, the “molecular stage” stage of a cancer is determined by detection of nucleic acid hypermethylation of one or more genes in a sample from the esophagus.

The term “subject” as used herein is meant to include vertebrates, preferably a mammal. Mammals include, but are not limited to, humans, camels, horses, goats, sheep, cows, dogs, cats, and the like.

The term “tumor” as used herein is intended to include an abnormal mass or growth of cells or tissue. A tumor can be benign or malignant.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9.

With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided herein.

### II. Oligonucleotide Probes

As used herein, “primer,” “probe,” and “oligonucleotide” are used interchangeably.

The term “nucleic acid probe” or a “probe specific for” a nucleic acid refers to a nucleic acid sequence that has at least about 80%, e.g., at least about 90%, e.g., at least about 95% contiguous sequence identity or homology to the nucleic acid sequence encoding the targeted sequence of interest. A probe (or oligonucleotide or primer) of the disclosure is at least about 8 nucleotides in length (e.g., at least about 8-50 nucleotides in length, e.g., at least about 10-40, e.g., at least about 15-35 nucleotides in length). The oligonucleotide probes or primers of the disclosure may comprise at least about eight nucleotides at the 3′ of the oligonucleotide that have at least about 80%, e.g., at least about 85%, e.g., at least about 90% contiguous identity to the targeted sequence of interest.

Primer pairs are useful for determination of the methylation status of a particular gene using PCR. The pairs of single-stranded DNA primers can be annealed to sequences within or surrounding the region of interest in order to prime amplifying DNA synthesis of the region itself. The first step of the process involves contacting a physiological sample obtained from a patient, which sample contains nucleic acid, with an oligonucleotide probe to form a hybridized DNA. The oligonucleotide probes that are useful in the methods of the present invention can be any probe comprised of between about 4 or 6 bases up to about 80 or 100 bases or more. In one embodiment of the present invention, the probes are between about 10 and about 20 bases.

In certain embodiments, the primers or probes of the present invention can be labeled using techniques known to those of skill in the art. For example, the labels used in the assays of disclosure can be primary labels (where the label comprises an element that is detected directly) or secondary labels (where the detected label binds to a primary label, e.g., as is common in immunological labeling). An introduction to labels (also called “tags”), tagging or labeling procedures, and detection of labels is found in Polak and Van Noorden (1997) Introduction to Immunocytochemistry, second edition, Springer Verlag, N.Y. and in Haugland (1996) Handbook of Fluorescent Probes and Research Chemicals, a combined handbook and catalogue Published by Molecular Probes, Inc., Eugene, Oreg. Primary and secondary labels can include undetected elements as well as detected elements. Useful primary and secondary labels in the present invention can include spectral labels such as fluorescent dyes (e.g., fluorescein and derivatives such as fluorescein isothiocyanate (FITC) and Oregon Green™, rhodamine and derivatives (e.g., Texas red, tetramethylrhodamine isothiocyanate (TRITC), etc.), digoxigenin, biotin, phycoerythrin, AMCA, CyDyes™, and the like), radiolabels (e.g., ³H, ¹²⁵I, ³⁵S, ¹⁴C, ³²P, ³³P), enzymes (e.g., horse-radish peroxidase, alkaline phosphatase) spectral colorimetric labels such as colloidal gold or colored glass or plastic (e.g., polystyrene, polypropylene, latex) beads. The label may be coupled directly or indirectly to a component of the detection assay (e.g., the labeled nucleic acid) according to methods well known in the art. As indicated above, a wide variety of labels may be used, with the choice of label depending on sensitivity required, ease of conjugation with the compound, stability requirements, available instrumentation, and disposal provisions.

In general, a detector that monitors a probe-substrate nucleic acid hybridization is adapted to the particular label that is used. Typical detectors include spectrophotometers, phototubes and photodiodes, microscopes, scintillation counters, cameras, film and the like, as well as combinations thereof. Examples of suitable detectors are widely available from a variety of commercial sources known to persons of skill. Commonly, an optical image of a substrate comprising bound labeled nucleic acids is digitized for subsequent computer analysis.

Examples of labels include those that use (1) chemiluminescence (using Horseradish Peroxidase and/or Alkaline Phosphatase with substrates that produce photons as breakdown products) with kits being available, e.g., from Molecular Probes, Amersham, Boehringer-Mannheim, and Life Technologies/Gibco BRL; (2) color production (using both Horseradish Peroxidase and/or Alkaline Phosphatase with substrates that produce a colored precipitate) (kits available from Life Technologies/Gibco BRL, and Boehringer-Mannheim); (3) hemifluorescence using, e.g., Alkaline Phosphatase and the substrate AttoPhos (Amersham) or other substrates that produce fluorescent products, (4) fluorescence (e.g., using Cy-5 (Amersham), fluorescein, and other fluorescent labels); (5) radioactivity using kinase enzymes or other end-labeling approaches, nick translation, random priming, or PCR to incorporate radioactive molecules into the labeled nucleic acid. Other methods for labeling and detection will be readily apparent to one skilled in the art.

Fluorescent labels can be used and have the advantage of requiring fewer precautions in handling, and being amendable to high-throughput visualization techniques (optical analysis including digitization of the image for analysis in an integrated system comprising a computer). Preferred labels are typically characterized by one or more of the following: high sensitivity, high stability, low background, low environmental sensitivity and high specificity in labeling. Fluorescent moieties, which are incorporated into the labels of the disclosure, are generally are known, including Texas red, dixogenin, biotin, 1- and 2-aminonaphthalene, p,p′-diaminostilbenes, pyrenes, quaternary phenanthridine salts, 9-aminoacridines, p,p′-diaminobenzophenone imines, anthracenes, oxacarbocyanine, merocyanine, 3-aminoequilenin, perylene, bis-benzoxazole, bis-p-oxazolyl benzene, 1,2-benzophenazin, retinal, bis-3-aminopyridinium salts, hellebrigenin, tetracycline, sterophenol, benzimidazolylphenylamine, 2-oxo-3-chromen, indole, xanthen, 7-hydroxycoumarin, phenoxazine, calicylate, strophanthidin, porphyrins, triarylmethanes, flavin and many others. Many fluorescent labels are commercially available from the SIGMA Chemical Company (Saint Louis, Mo.), Molecular Probes, R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka ChemicaBiochemika Analytika (Fluka Chemie AG, Buchs, Switzerland), and Applied Biosystems™ (Foster City, Calif.), as well as many other commercial sources known to one of skill.

Means of detecting and quantifying labels are well known to those of skill in the art. Thus, for example, where the label is a radioactive label, means for detection include a scintillation counter or photographic film as in autoradiography. Where the label is optically detectable, typical detectors include microscopes, cameras, phototubes and photodiodes and many other detection systems that are widely available.

Oligonucleotide probes may be prepared having any of a wide variety of base sequences according to techniques that are well known in the art. Suitable bases for preparing the oligonucleotide probe may be selected from naturally occurring nucleotide bases such as adenine, cytosine, guanine, uracil, and thymine; and non-naturally occurring or “synthetic” nucleotide bases such as 7-deaza-guanine 8-oxo-guanine, 6-mercaptoguanine, 4-acetylcytidine, 5-(carboxyhydroxyethyl)uridine, 2′-0-methylcytidine, 5-carboxymethylamino-methyl-2-thioridine, 5-carboxymethylaminomethyluridine, dihydrouridine, 2′-0-methylpseudouridine, D-galactosylqueosine, 2′-0-methylguanosine, inosine, N6-isopentenyladenosine, 1-methyladenosine, 1-methylpseeudouridine, 1-methylguanosine, 1-methylinosine, 2,2-dimethylguanosine, 2-methyladenosine, 2-methylguanosine, 3-methy1cytidine, 5-methylcytidine, N6-methyladenosine, 7-methylguanosine, 5-methylamninomethyluridine, 5-methoxyaminomethyl-2-thiouridine, D-mannosylqueosine, 5-methloxycarbonylmethyluridine, 5-methoxyuridine, 2-methyltio-N6-isopentenyladenosine, N-((9-D-ribofuranosy 1-2-methylthiopurine-6-yl)carbamoyl)threonine, N-((9-D-ribofuranosylpurine-6-yl)N-methyl-carbamoyl)threonine, uridine-5-oxyacetic acid methylester, uridine-5-oxyacetic acid, wybutoxosine, pseudouridine, queosine, 2-thiocytidine, 5-methyl-2-thiouridine, 2-thiouridine, 2-thiouridine, 5-Methylurdine, N-((9-beta-D-ribofuranosylpurine-6-yl)carbamoyl)threonine, 2′-0-methyl-5-methyluridine, 2′-0-methylurdine, wybutosine, and 3-(3-amino-3-carboxypropyl)uridine. Any oligonucleotide backbone may be employed, including DNA, RNA (although RNA is less preferred than DNA), modified sugars such as carbocycles, and sugars containing 2′ substitutions such as fluoro and methoxy. The oligonucleotides may be oligonucleotides wherein at least one, or all, of the internucleotide bridging phosphate residues are modified phosphates, such as methyl phosphonates, methyl phosphonotlioates, phosphoroinorpholidates, phosphoropiperazidates and phosplioramidates (for example, every other one of the internucleotide bridging phosphate residues may be modified as described). The oligonucleotide may be a “peptide nucleic acid” such as described in Nielsen et al., Science, 254: 1497-1500 (1991).

The only requirement is that the oligonucleotide probe should possess a sequence at least a portion of which is capable of binding to a known portion of the sequence of the DNA sample. The nucleic acid probes provided by the present invention are useful for a number of purposes.

### III. Detection of Methylation

In higher order eukaryotes, DNA is methylated only at cytosines located 5′ to guanosine in the CpG dinucleotide. This modification has important regulatory effects on gene expression, especially when involving CpG rich areas, known as CpG islands, located in the promoter regions of many genes. While almost all gene-associated islands are protected from methylation on autosomal chromosomes, extensive methylation of CpG islands has been associated with transcriptional inactivation of selected imprinted genes and genes on the inactive X-chromosome of females. Aberrant methylation of normally unmethylated CpG islands has been described as a frequent event in immortalized and transformed cells, and has been associated with transcriptional inactivation of defined tumor suppressor genes in human cancers. Any method that is sufficient to detect methylation is a suitable for use in the methods of the invention. Any method that is sufficient to detect hypermethylation, e.g., a method that can detect methylation of nucleotides at levels as low as 0.10%, is a suitable for use in the methods of the invention.

Methylation-on-Beads is a single-tube method for polynucleotide extraction and bisulfite conversion that provides a rapid and highly efficient method for DNA extraction, bisulfite treatment and detection of DNA methylation using silica superparamagnetic particles (SSP). All steps are implemented without centrifugation or air drying that provides superior yields relative to conventional methods for DNA extraction and bisulfite conversion. SSP serve as solid substrate for DNA binding throughout the multiple stages of each process. Specifically, SSP are first used to capture genomic DNA from raw tissue samples, processed tissue samples or cultured cells. Sodium bisulfite treatment is then carried out in the presence of SSP without tube transfers. Finally, the bisulfite treated DNA is analyzed to determine the methylation status, Methylation-on-Beads allows for convenient, efficient and contamination-resistant methylation detection in a single tube or other reaction platform. Methods for carrying out methylation-on-beads are known in the art, and described, for example, in PCT/US2009/000039, which is incorporated herein in its entirety.

According to the techniques herein, PCR analysis is preferred, and more particularly, methylation-specific PCR analysis, for example qualitative methylation specific PCR (QMSP). Other methods that can be used include, but are not limited to, bisulfate modification to identify changes in DNA methylation of the genes including, but not limited to, ZNF542, ZNF132, cg20655070, TAC1 and SLC35F1. This correlates with loss of expression. Additional methods to determine the methylation status of this gene include genomic bisulfite sequencing. MassSPEC methods of methylation detection, and those relying on methylation sensitive restriction digestion of DNA or methyl binding proteins. Other methods which examine loss of expression of the gene, for example RT-PCR approaches, or protein expression, for example immunohistochemistry or western blot analysis, might also be used to determine inactivation of genes including, but not limited to, ZNF542, ZNF132, cg20655070, TAC1 and SLC35F1 and thus risk of developing ESCC.

In particular embodiments, hypermethylation is detected using quantitative methylation specific polymerase chain reaction (QMSP). In further embodiments, QMSP can be combined with methylation on beads (MOB).

Methylation-sensitive restriction endonucleases can be used to detect methylated CpG dinucleotide motifs. Such endonucleases may either preferentially cleave methylated recognition sites relative to non-methylated recognition sites or preferentially cleave non-methylated relative to methylated recognition sites. Examples of the former are Acc III, Ban I, BstN I, Msp I, and Xma I. Examples of the latter are Acc II, Ava I, BssH II, BstU I, Hpa I, and Not I. Alternatively, chemical reagents can be used which selectively modify either the methylated or non-methylated form of CpG dinucleotide motifs.

Modified products can be detected directly, or after a further reaction which creates products which are easily distinguishable. Techniques that detect altered size and/or charge can be used to detect modified products, including but not limited to electrophoresis, chromatography, and mass spectrometry. Other techniques that are reliant on specific sequences can be used, including but not limited to hybridization, amplification, sequencing, and ligase chain reaction. Combinations of such techniques can be uses as is desired. Examples of such chemical reagents for selective modification include hydrazine and bisulfite ions. Hydrazine-modified DNA can be treated with piperidine to cleave it. Bisulfite ion-treated DNA can be treated with alkali.

Other techniques that can be used include technologies suitable for detecting DNA methylation with the use of bisulfite treatment include MSP, Mass Array, MethylLight, QAMA (quantitative analysis of methylated alleles), ERMA (enzymatic regional methylation assay), HeavyMethyl, pyrosequencing technology, MS-SNuPE, Methylquant, oligonucleotide-based microarray.

The ability to monitor the real-time progress of the PCR changes the way that PCR-based quantification of DNA and RNA may be approached. Reactions are characterized by the point in time during cycling when amplification of a PCR product is first detected rather than the amount of PCR product accumulated after a fixed number of cycles. The higher the starting copy number of the nucleic acid target, the sooner a significant increase in fluorescence is observed. An amplification plot is the plot of fluorescence signal versus cycle number. In the initial cycles of PCR, there is little change in fluorescence signal. This defines the baseline for the amplification plot. An increase in fluorescence above the baseline indicates the detection of accumulated PCR product. A fixed fluorescence threshold can be set above the baseline. The parameter C_{T} (threshold cycle) is defined as the fractional cycle number at which the fluorescence passes the fixed threshold. For example, the PCR cycle number at which fluorescence reaches a threshold value of 10 times the standard deviation of baseline emission may be used as C_{T} and it is inversely proportional to the starting amount of target cDNA. A plot of the log of initial target copy number for a set of standards versus C_{T} is a straight line. Quantification of the amount of target in unknown samples is accomplished by measuring C_{T} and using the standard curve to determine starting copy number.

The entire process of calculating C_{TS}, preparing a standard curve, and determining starting copy number for unknowns can be performed by software, for example that of the 7700 system or 7900 system of Applied Biosystems. Real-time PCR requires an instrumentation platform that consists of a thermal cycler, computer, optics for fluorescence excitation and emission collection, and data acquisition and analysis software. These machines, available from several manufacturers, differ in sample capacity (some are 96-well standard format, others process fewer samples or require specialized glass capillary tubes), method of excitation (some use lasers, others broad spectrum light sources with tunable filters), and overall sensitivity. There are also platform-specific differences in how the software processes data. Real-time PCR machines are available at core facilities or labs that have the need for high throughput quantitative analysis.

Briefly, in the Q-PCR method the number of target gene copies can be extrapolated from a standard curve equation using the absolute quantitation method. For each gene, cDNA from a positive control is first generated from RNA by the reverse transcription reaction. Using about 1 μl of this cDNA, the gene under investigation is amplified using the primers by means of a standard PCR reaction. The amount of amplicon obtained is then quantified by spectrophotometry and the number of copies calculated on the basis of the molecular weight of each individual gene amplicon. Serial dilutions of this amplicon are tested with the Q-PCR assay to generate the gene specific standard curve. Optimal standard curves are based on PCR amplification efficiency from 90 to 100% (100% meaning that the amount of template is doubled after each cycle), as demonstrated by the slope of the standard curve equation. Linear regression analysis of all standard curves should show a high correlation (R² coefficient 0.98). Genomic DNA can be similarly quantified.

When measuring transcripts of a target gene, the starting material, transcripts of a housekeeping gene are quantified as an endogenous control. Beta-actin is one of the most used nonspecific housekeeping genes. For each experimental sample, the value of both the target and the housekeeping gene are extrapolated from the respective standard curve. The target value is then divided by the endogenous reference value to obtain a normalized target value independent of the amount of starting material.

The above-described quantitative real-time PCR methodology has been adapted to perform quantitative methylation-specific PCR (QMSP) by utilizing the external primers pairs in round one (multiplex) PCR and internal primer pairs in round two (real time MSP) PCR. Thus each set of genes has one pair of external primers and two sets of three internal primers/probes (internal sets are specific for unmethylated or methylated DNA). The external primer pairs can co-amplify a cocktail of genes, each pair selectively hybridizing to a member of the panel of genes being investigated using the invention method. The method of methylation-specific PCR (QMSP) has been described in US Patent Application 20050239101, incorporated by reference in its entirety herein.

Methylation can be detected using two-stage, or “nested” PCR, for example as described in U.S. Pat. No. 7,214,485, incorporated by reference in its entirety herein. For example, two-stage, or “nested” polymerase chain reaction method is disclosed for detecting methylated DNA sequences at sufficiently high levels of sensitivity to permit cancer screening in biological fluid samples, such as e.g., sputum, obtained non-invasively.

A method for assessing the methylation status of any group of CpG sites within a CpG island, independent of the use of methylation-sensitive restriction enzymes, is described in U.S. Pat. No. 6,017,704, which is incorporated by reference in its entirety herein and described briefly as follows. This method employs primers that specific for the bisulfite reaction such that the PCR reaction itself is used to distinguish between the chemically modified methylated and unmethylated DNA, which adds an improved sensitivity of methylation detection. Unlike previous genomic sequencing methods for methylation identification which utilizes amplification primers which are specifically designed to avoid the CpG sequences. QMSP primers themselves are specifically designed to recognize CpG sites to take advantage of the differences in methylation to amplify specific products to be identified by the invention assay. The methods of QMSP include modification of DNA by sodium bisulfite or a comparable agent that converts all unmethylated but not methylated cytosines to uracil, and subsequent amplification with primers specific for methylated versus unmethylated DNA. This method of “methylation specific PCR (MSP)” requires only small amounts of DNA, is sensitive to 0.10% of methylated alleles of a given CpG island locus, and can be performed on DNA extracted from paraffin-embedded samples, for example. In addition, MSP eliminates the false positive results inherent to previous PCR-based approaches which relied on differential restriction enzyme cleavage to distinguish methylated from unmethylated DNA.

MSP provides significant advantages over previous PCR and other methods used for assaying methylation. MSP is markedly more sensitive than Southern analyses, facilitating detection of low numbers of methylated alleles and the study of DNA from small samples. MSP allows the study of paraffin-embedded materials, which could not previously be analyzed by Southern analysis. MSP also allows examination of all CpG sites, not just those within sequences recognized by methylation-sensitive restriction enzymes. This markedly increases the number of such sites which can be assessed and will allow rapid, fine mapping of methylation patterns throughout CpG rich regions. MSP also eliminates the frequent false positive results due to partial digestion of methylation-sensitive enzymes inherent in previous PCR methods for detecting methylation. Furthermore, with MSP, simultaneous detection of unmethylated and methylated products in a single sample confirms the integrity of DNA as a template for PCR and allows a semi-quantitative assessment of allele types which correlates with results of Southern analysis. Finally, the ability to validate the amplified product by differential restriction patterns is an additional advantage.

MSP may provide information similar to genomic sequencing, but can be performed with some advantages as follows. MSP is simpler and requires less time than genomic sequencing, with a typical PCR and gel analysis taking 4-6 hours. In contrast, genomic sequencing, amplification, cloning, and subsequent sequencing may take days. MSP also avoids the use of expensive sequencing reagents and the use of radioactivity. Both of these factors make MSP better suited for the analysis of large numbers of samples. The use of PCR as the step to distinguish methylated from unmethylated DNA in MSP allows for significant increase in the sensitivity of methylation detection. For example, if cloning is not used prior to genomic sequencing of the DNA, less than 10% methylated DNA in a background of unmethylated DNA cannot be seen (Myohanen, et al., supra). The use of PCR and cloning does allow sensitive detection of methylation patterns in very small amounts of DNA by genomic sequencing (Frommer, et al., Proc. Natl. Acad. Sci. USA, 89:1827, 1992; Clark, et al., Nucleic Acids Research, 22:2990, 1994). However, this means in practice that it would require sequencing analysis of 10 clones to detect 10% methylation, 100 clones to detect 1% methylation, and to reach the level of sensitivity demonstrated with MSP (1:1000) according to the techniques, one would have to sequence 1000 individual clones.

“Multiplex methylation-specific PCR” is a unique version of methylation-specific PCR. Methylation-specific PCR is described in U.S. Pat. Nos. 5,786,146; 6,200,756; 6,017,704 and 6,265,171, each of which is incorporated herein by reference in its entirety. Multiplex methylation-specific PCR utilizes MSP primers for a multiplicity of markers, for example three or more different markers, in a two-stage nested PCR amplification reaction. The primers used in the first PCR reaction are selected to amplify a larger portion of the target sequence than the primers of the second PCR reaction. The primers used in the first PCR reaction are referred to herein as “external primers” or “DNA primers” and the primers used in the second PCR reaction are referred to herein as “MSP primers.” Two sets of primers (i.e., methylated and unmethylated for each of the markers targeted in the reaction) are used as the MSP primers. In addition in multiplex methylation-specific PCR, as described herein, a small amount (i.e., 1 μl) of a 1:10 to about 106 dilution of the reaction product of the first “external” PCR reaction is used in the second “internal” MSP PCR reaction.

The term “primer” as used herein refers to a sequence comprising two or more deoxyribonucleotides or ribonucleotides, preferably more than three, and most preferably more than 8, which sequence is capable of initiating synthesis of a primer extension product, which is substantially complementary to a polymorphic locus strand. Environmental conditions conducive to synthesis include the presence of nucleoside triphosphates and an agent for polymerization, such as DNA polymerase, and a suitable temperature and pH. The primer is preferably single stranded for maximum efficiency in amplification, but may be double stranded, if double stranded, the primer is first treated to separate its strands before being used to prepare extension products. Preferably, the primer is an oligodeoxy ribonucleotide. The primer must be sufficiently long to prime the synthesis of extension products in the presence of the inducing agent for polymerization. The exact length of primer will depend on many factors, including temperature, buffer, and nucleotide composition. The oligonucleotide primer typically contains 12-20 or more nucleotides, although it may contain fewer nucleotides.

Primers of the invention are designed to be “substantially” complementary to each strand of the oligonucleotide to be amplified and include the appropriate G or C nucleotides as discussed above. This means that the primers must be sufficiently complementary to hybridize with their respective strands under conditions that allow the agent for polymerization to perform. In other words, the primers should have sufficient complementarity with a 5′ and 3′ oligonucleotide to hybridize therewith and permit amplification of CpG containing nucleic acid sequence.

Primers of the invention are employed in the amplification process, which is an enzymatic chain reaction that produces exponentially increasing quantities of target locus relative to the number of reaction steps involved (e.g., polymerase chain reaction or PCR). Typically, one primer is complementary to the negative (−) strand of the locus (antisense primer) and the other is complementary to the positive (+) strand (sense primer). Annealing the primers to denatured nucleic acid followed by extension with an enzyme, such as the large fragment of DNA Polymerase I (Klenow) and nucleotides, results in newly synthesized + and − strands containing the target locus sequence. Because these newly synthesized sequences are also templates, repeated cycles of denaturing, primer annealing, and extension results in exponential production of the region (i.e., the target locus sequence) defined by the primer. The product of the chain reaction is a discrete nucleic acid duplex with termini corresponding to the ends of the specific primers employed.

The oligonucleotide primers used in invention methods may be prepared using any suitable method, such as conventional phosphotriester and phosphodiester methods or automated embodiments thereof. In one such automated embodiment, diethylphos-phoramidites are used as starting materials and may be synthesized as described by Beaucage, et al. (Tetrahedron Letters, 22:1859-1862, 1981). One method for synthesizing oligonucleotides on a modified solid support is described in U.S. Pat. No. 4,458,066.

In certain preferred embodiments, methylation of genes including, but not limited to, ZNF542, ZNF132, cg20655070, TAC1 and SLC35F1, may be determined by real-time MSP using molecular beacons. The method consists in certain embodiments of using a gene for normalization, e.g., ACTB.

The primers used in the invention for amplification of the CpG-containing nucleic acid in the specimen, after bisulfite modification, specifically distinguish between untreated or unmodified DNA, methylated, and non-methylated DNA. QMSP primers for the non-methylated DNA preferably have a T in the 3′ CG pair to distinguish it from the C retained in methylated DNA, and the complement is designed for the antisense primer. MSP primers usually contain relatively few Cs or Gs in the sequence since the Cs will be absent in the sense primer and the Gs absent in the antisense primer (C becomes modified to U (uracil) which is amplified as T (thymidine) in the amplification product).

An additional method of determining the results after sodium bisulfite treatment would be to sequence the DNA to directly observe any bisulfite-modifications. Pyrosequencing technology is a method of sequencing-by-synthesis in real time. It is based on an indirect bioluminometric assay of the pyrophosphate (PPi) that is released from each deoxynucleotide (dNTP) upon DNA-chain elongation. This method presents a DNA template-primer complex with a dNTP in the presence of an exonuclease-deficient Klenow DNA polymerase. The four nucleotides are sequentially added to the reaction mix in a predetermined order. If the nucleotide is complementary to the template base and thus incorporated, PPi is released. The PPi and other reagents are used as a substrate in a luciferase reaction producing visible light that is detected by either a luminometer or a charge-coupled device. The light produced is proportional to the number of nucleotides added to the DNA primer and results in a peak indicating the number and type of nucleotide present in the form of a pyrogram. Pyrosequencing can exploit the sequence differences that arise following sodium bisulfite-conversion of DNA.

A variety of amplification techniques may be used in a reaction for creating distinguishable products. Some of these techniques employ PCR. Other suitable amplification methods include the ligase chain reaction (LCR) (Barringer et al, 1990), transcription amplification (Kwoh et al. 1989; WO88/10315), selective amplification of target polynucleotide sequences (U.S. Pat. No. 6,410,276), consensus sequence primed polymerase chain reaction (U.S. Pat. No. 4,437,975), arbitrarily primed polymerase chain reaction (WO90/06995), nucleic acid based sequence amplification (NASBA) (U.S. Pat. Nos. 5,409,818; 5,554,517; 6,063,603), nick displacement amplification (WO2004/067726).

Sequence variation that reflects the methylation status at CpG dinucleotides in the original genomic DNA offers two approaches to PCR primer design. In the first approach, the primers do not themselves “cover” or hybridize to any potential sites of DNA methylation; sequence variation at sites of differential methylation are located between the two primers. Such primers are used in bisulphite genomic sequencing, COBRA, Ms-SNuPE. In the second approach, the primers are designed to anneal specifically with either the methylated or unmethylated version of the converted sequence. If there is a sufficient region of complementarity, e.g., 12, 15, 18, or 20 nucleotides, to the target, then the primer may also contain additional nucleotide residues that do not interfere with hybridization but may be useful for other manipulations. Exemplary of such other residues may be sites for restriction endonuclease cleavage, for ligand binding or for factor binding or linkers or repeats. The oligonucleotide primers may or may not be such that they are specific for modified methylated residues.

One way to distinguish between modified and unmodified DNA is to hybridize oligonucleotide primers which specifically bind to one form or the other of the DNA. After hybridization, an amplification reaction can be performed and amplification products assayed. The presence of an amplification product indicates that a sample hybridized to the primer. The specificity of the primer indicates whether the DNA had been modified or not, which in turn indicates whether the DNA had been methylated or not. For example, bisulfite ions modify non-methylated cytosine bases, changing them to uracil bases. Uracil bases hybridize to adenine bases under hybridization conditions. Thus an oligonucleotide primer which comprises adenine bases in place of guanine bases would hybridize to the bisulfite-modified DNA, whereas an oligonucleotide primer containing the guanine bases would hybridize to the non-modified (methylated) cytosine residues in the DNA. Amplification using a DNA polymerase and a second primer yield amplification products which can be readily observed. Such a method is termed MSP (Methylation Specific PCR; U.S. Pat. Nos. 5,786,146; 6,017,704; 6,200,756). The amplification products can be optionally hybridized to specific oligonucleotide probes which may also be specific for certain products. Alternatively, oligonucleotide probes can be used which will hybridize to amplification products from both modified and nonmodified DNA.

Another way to distinguish between modified and nonmodified DNA is to use oligonucleotide probes which may also be specific for certain products. Such probes can be hybridized directly to modified DNA or to amplification products of modified DNA. Oligonucleotide probes can be labeled using any detection system known in the art. These include but are not limited to fluorescent moieties, radioisotope labeled moieties, bioluminescent moieties, luminescent moieties, chemiluminescent moieties, enzymes, substrates, receptors, or ligands.

Still another way for the identification of methylated CpG dinucleotides utilizes the ability of the MBD domain of the McCP2 protein to selectively bind to methylated DNA sequences (Cross et al, 1994; Shiraishi et al, 1999). Restriction endonuclease digested genomic DNA is loaded onto expressed His-tagged methyl-CpG binding domain that is immobilized to a solid matrix and used for preparative column chromatography to isolate highly methylated DNA sequences.

Real time chemistry allows for the detection of PCR amplification during the early phases of the reactions, and makes quantitation of DNA and RNA easier and more precise. A few variations of the real-time PCR are known. They include the TAQMAN® system and Molecular Beacon system which have separate probes labeled with a fluorophore and a fluorescence quencher. In the SCORPION® system the labeled probe in the form of a hairpin structure is linked to the primer.

DNA methylation analysis has been performed successfully with a number of techniques which include the MALDI-TOFF, MassARRAY, MethyLight, Quantitative analysis of ethylated alleles (QAMA), enzymatic regional methylation assay (ERMA), HeavyMethyl, QBSUPT, MS-SNuPE, MethylQuant, Quantitative PCR sequencing, and Oligonucleotide-based microarray systems.

The number of genes whose silencing is tested and/or detected can vary: one, two, three, four, five, or more genes can be tested and/or detected. In some examples, methylation of at least one gene is detected. In other examples, methylation of at least two genes is detected. However, methylation of any number of genes may be detected, using the methods as described herein.

For purposes of the invention, an antibody or nucleic acid probe specific for a gene or gene product may be used to detect the presence of methylation either by detecting the level of polypeptide (using antibody) or methylation of the polynucleotide (using nucleic acid probe) in biological fluids or tissues. For antibody-based detection, the level of the polypeptide is compared with the level of polypeptide found in a corresponding “normal” tissue. Oligonucleotide primers based on any coding sequence region of the promoter of the following genes ZNF542, ZNF132, cg20655070, TAC1 and SLC35F1.

In particular embodiments, oligonucleotide primers are based on the coding sequence region of the promoter in genes including, but not limited to, ZNF542, ZNF132, cg20655070, TAC1 and SLC35F1, and are useful for amplifying DNA, for example by PCR. These genes are merely listed as examples and are not meant to be limiting.

Any of the methods as described herein can be used in high throughput analysis of DNA methylation. For example, U.S. Pat. No. 7,144,701, incorporated by reference in its entirety herein, describes differential methylation hybridization (DMH) for a high-throughput analysis of DNA methylation.

### IV. Methods for Using the Gene Panels

The detection of hypermethylation as described herein can be used to detect or diagnose a proliferative disease. In particular embodiments, the methods comprise using bisulfite treated DNA. In certain embodiments, the detection of hypermethylation as described in these methods can be used after surgery or therapy to treat a proliferative disease. In other embodiments, the detection of methylation as described in these methods can be used to predict the recurrence of a proliferative disease. The detection of methylation as described in these methods can be used to stage a proliferative disease. In further embodiments, the detection of methylation as described in these methods can be used to determine a course of treatment for a subject. These embodiments are discussed in further detail herein.

The methods of the invention as described herein are used in certain exemplary embodiments to identify ESCC by detecting hypermethylation of one or more genes including ZNF542, ZNF132, cg20655070, TAC1 and SLC35F1 in one or more samples. In this way, the detection of nucleic acid hypermethylation identifies ESCC.

The methods of the invention can be used to predict risk of developing ESCC in a subject. In preferred embodiments, the method comprises detecting nucleic acid methylation of one or more genes including ZNF542, ZNF132, cg20655070, TACT and SLC35F1 in one or more samples, and wherein detecting nucleic acid methylation identifies risk of developing cancer in a subject.

The present invention features methods for identifying a subject that will respond to one or more ESCC-directed therapies. In preferred embodiments, the methods comprise detecting nucleic acid methylation of certain genes, for example, one or more of ZNF542, ZNF132, cg20655070, TACT and SLC35F1, in one or more samples, wherein detecting nucleic acid methylation identifies a subject that will respond to one or more ESCC-directed therapies.

The methods described herein may be used to determine a course of treatment for a subject. These methods comprise extracting nucleic acid from one or more cell or tissue samples, detecting nucleic acid methylation of one or more genes including ZNF542, ZNF132, cg20655070, TACT and SLC35F1, in the sample, wherein nucleic acid methylation of one or more of ZNF542, ZNF132, cg20655070, TACT and SLC35F1 genes indicates the subject is at risk of developing ESCC.

The conditions associated with aberrant methylation of genes that can be detected or monitored specifically include ESCC. The panels described herein can also be used to detect other conditions including, but are not limited to, metastases associated with carcinomas and sarcomas of all kinds, including one or more specific types of cancer, e.g., a lung cancer, breast cancer, an alimentary or gastrointestinal tract cancer such as colon, esophageal and pancreatic cancer, a liver cancer, a skin cancer, an ovarian cancer, an endometrial cancer, a prostate cancer, a lymphoma, hematopoietic tumors, such as a leukemia, a kidney cancer, a bronchial cancer, a muscle cancer, a bone cancer, a bladder cancer or a brain cancer, such as astrocytoma, anaplastic astrocytoma, glioblastoma, medulloblastoma, and neuroblastoma and their metastases. Suitable pre-malignant lesions to be detected or monitored using the invention include, but are not limited to, lobular carcinoma in situ and ductal carcinoma in situ.

### V. Methods of Monitoring or Treatment

The invention as described herein may be used to treat a subject having or at risk for having cancer (e.g., ESCC). Accordingly, the method comprises identifying nucleic acid methylation of one or more genes including, but not limited to, ZNF542, ZNF132, cg20655070, TACT and SLC35F1. The method can further comprise the step of performing an endoscopy on the subject. The method can be used in combination with one or more ESCC-directed therapies. In a specific embodiment, the method can further comprise administering to the subject a therapeutically effective amount of a demethylating agent, thereby treating a subject having or at risk for having cancer. Demethylating agents include, but are not limited to, 5-aza-2′-deoxycytidine, 5-aza-cytidine, Zebularine, procaine, L-ethionine, 5-azadeoxycytidine (DAC) SGI-110 (guadecitabine) or analogs of the foregoing. In a specific embodiment, the demethylating agent comprises AZA.

In a specific embodiment, the treatment step comprises a cisplatin- and 5-fluorouacil (CF)-based regimen. Chemoradiotherapy (CRT) is the standard treatment for unresectable ESCC and is also an option for resectable tumors. For patients who are inoperable, concurrent CRT can be administered. Docetaxil, cisplatin and 5-fluorouracil (DCF) therapy can be used with or without radiotherapy.

In one embodiment, the treatment step comprises endoscopic resection. In another embodiment, a subject is treated with surgery to remove the cancer. In another embodiment, the treatment step comprises transthoracic esophagectomy. In yet another embodiment, the treatment step comprises transhiatal esophagectomy. In further embodiments, a multimodal therapy approach is used. For example, neoadjuvant chemotherapy can be administered prior to surgery. Alternatively, chemotherapy or chemoradiotherapy (CRT) is administered.

In certain embodiments, the treatment step can include endoscopy and dilation, endoscopy with stent placements, electrocoagulation or cryotherapy.

The subject can also be treated with targeted therapy. In one embodiment, the therapy comprises HER2-targeted therapy including trastuzumab (Herceptin, Ogivri). HER2-targeted therapy can be used along with chemotherapy. In another embodiment, the therapy comprises anti-angiogenesis therapy including, but not limited to, ramucirumab (Cyramza). Remucirumab can be administered by itself or with paclitaxel (Abraxane).

In other embodiments, immunotherapy can be used. In further embodiments, a check point inhibitor can be administered. The checkpoint inhibitor can include, but is not limited to, an anti-PD1 antibody (e.g., nivolumab, pembrolizumab (keytruda)), an anti-PDL-1 antibody (e.g., Medi4736) or an anti-CTLA4 antibody (e.g., tremelimumab). In other embodiments, an HDAC inhibitor can be used. The HDAC inhibitor can include, but is not limited to, givinostat, entinostat or analogs thereof. In other embodiments, an ESCC therapy can comprises ipilimumab (Yervoy). In yet another embodiment, the therapy can comprise durvalumab, chemotherapy and radiation therapy prior to surgery. Nivolumab and Ipiplumab can be administered to the subject. In other embodiments, afatinib dimaleate and paclitaxel can be used. In another embodiment, nivolumab can be combined with fluorouracil and cisplatin.

In further embodiments, the method can be used in combination with one or more chemotherapeutic agents. Anti-cancer drugs that may be used in the various embodiments of the invention, including pharmaceutical compositions and dosage forms and kits of the invention, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; erlotinib; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gefitinib; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine, mechlorethamine oxide hydrochloride rethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; navelbine; nivolumab; nocodazole; nogalamycin; ormaplatin; oxisuran; paclitaxel; pegaspargase; peliomycin; pemetrexed; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, improsulfan, benzodepa, carboquone, triethylenemelamine, triethylenephosphoramide, triethylenethiophosphoramide, trimethylolomelamine, chlomaphazine, novembichin, phenesterine, trofosfamide, estermustine, chlorozotocin, gemzar, nimustine, ranimustine, dacarbazine, mannomustine, mitobronitol, aclacinomycins, actinomycin F(1), azaserine, bleomycin, carubicin, carzinophilin, chromomycin, daunorubicin, daunomycin, 6-diazo-5-oxo-1-norleucine, doxorubicin, olivomycin, plicamycin, porfiromycin, puromycin, tubercidin, zorubicin, denopterin, pteropterin, 6-mercaptopurine, ancitabine, 6-azauridine, carmofur, cytarabine, dideoxyuridine, enocitabine, pulmozyme, aceglatone, aldophosphamide glycoside, bestrabucil, defofamide, demecolcine, elfornithine, elliptinium acetate, etoglucid, flutamide, hydroxyurea, lentinan, phenamet, podophyllinic acid, 2-ethylhydrazide, razoxane, spirogermanium, tamoxifen, taxotere, tenuazonic acid, triaziquone, 2,2′,2″-trichlorotriethylamine, urethan, vinblastine, vincristine, vindesine and related agents. 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cisporphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; taxel; taxel analogues; taxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer, Preferred additional anti-cancer drugs are 5-fluorouracil and leucovorin, Additional cancer therapeutics include monoclonal antibodies such as rituximab, trastuzumab and cetuximab.

Another way to restore epigenetically silenced gene expression is to introduce a non-methylated polynucleotide into a cell, so that it will be expressed in the cell. Various gene therapy vectors and vehicles are known in the art and any can be used as is suitable for a particular situation. Certain vectors are suitable for short term expression and certain vectors are suitable for prolonged expression. Certain vectors are trophic for certain organs and these can be used as is appropriate in the particular situation. Vectors may be viral or non-viral. The polynucleotide can, but need not, be contained in a vector, for example, a viral vector, and can be formulated, for example, in a matrix such as a liposome, microbubbles. The polynucleotide can be introduced into a cell by administering the polynucleotide to the subject such that it contacts the cell and is taken up by the cell and the encoded polypeptide expressed. Preferably the specific polynucleotide will be one which the patient has been tested for and been found to carry a silenced version.

### VI. Kits

The invention features kits for identifying the nucleic acid methylation state of genes including, but not limited to, ZNF542, ZNF132, cg20655070, TAC1 and SLC35F1 comprising gene specific primers for use in polymerase chain reaction (PCR), and instructions for use.

A kit as described herein can include at least one first nucleic acid primer (e.g., at least 8 nucleotides in length) that is complementary to a bisulfite-converted nucleic acid sequence comprising a CpG dinucleotide in the ZNF542 gene. In some embodiments, the at least one first nucleic acid primer detects the methylated CpG dinucleotide. The kit comprises primers for ZNF542 including one or more of SEQ ID NOS: 1-3.

In some embodiments, a kit further can include at least one first nucleic acid primer (e.g., at least 8 nucleotides in length) that is complementary to a bisulfite-converted nucleic acid sequence comprising a CpG dinucleotide in the ZNF132 gene, where the at least one second nucleic acid primer detects the methylated CpG dinucleotide. In a specific embodiment, the kit comprises primers for ZNF132 including one or more of SEQ ID NOS:4-6.

A kit as described herein can include at least one first nucleic acid primer (e.g., at least 8 nucleotides in length) that is complementary to a bisulfite-converted nucleic acid sequence comprising a CpG dinucleotide in the cg20655070 gene, where the at least one first nucleic acid primer detects the methylated CpG dinucleotide. In a specific embodiment, the kit comprises primers for cg20655070 including one or more of SEQ ID NOS:7-9.

In some embodiments, a kit further can include at least one first nucleic acid primer (e.g., at least 8 nucleotides in length) that is complementary to a bisulfite-converted nucleic acid sequence comprising a CpG dinucleotide in the TAC1 gene, where the at least one second nucleic acid primer detects the methylated CpG dinucleotide. In particular embodiments, the kit comprises primers for TAC1 including one or more of SEQ ID NOS:10-12.

A kit as described herein can include at least one first nucleic acid primer (e.g., at least 8 nucleotides in length) that is complementary to a bisulfite-converted nucleic acid sequence comprising a CpG dinucleotide in the SLC35F1 gene, where the at least one first nucleic acid primer detects the methylated CpG dinucleotide. In a specific embodiment, the kit comprises primers for SLC35F1 including one or more of SEQ ID NOS:13-15.

It would be appreciated that any of the nucleic acid primers, probes or oligonucleotides described herein can include one or more nucleotide analogs and/or one or more synthetic or non-natural nucleotides.

It also would be appreciated that any of the kits described herein can include a solid substrate. In some embodiments, one or more of the nucleic acid primers can be bound to the solid support. Examples of solid supports include, without limitation, polymers, glass, semiconductors, papers, metals, gels or hydrogels. Additional examples of solid supports include, without limitation, microarrays or microfluidics cards.

It also would be appreciated that any of the kits described herein can include one or more detectable labels. In some embodiments, one or more of the nucleic acid primers can be labeled with the one or more detectable labels. Representative detectable labels include, without limitation, an enzyme label, a fluorescent label, and a colorimetric label.

As described above, the PCR, in particularly preferred examples, is quantitative methylation specific PCR (QMSP). In some embodiments, QMSP is combined with methylation on beads (MOB). In particular embodiments, the kit comprises the reagents necessary for the methylation on beads protocol described herein.

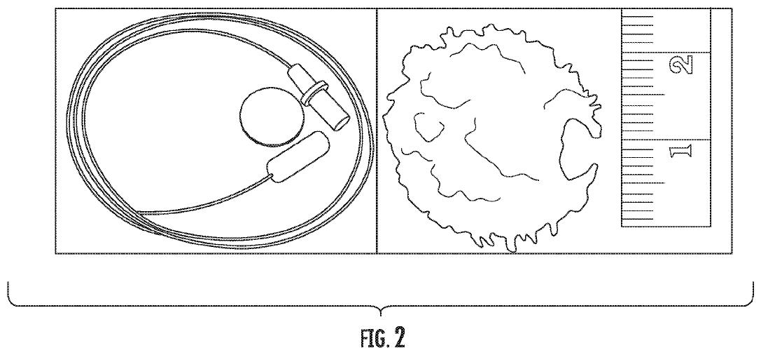

In particular embodiments, the kit comprises a device for retrieving cell samples from the esophagus. In a specific embodiment, the device is a retrievable sponge. In a more specific embodiment, the device is the EsophaCap™ swallowable sponge. See FIG. 2. The kit can further comprise a container for storing the retrieved sponge. The container can comprise a preservative solution to support cells during transport. In one embodiment, the container contains an alcohol-based buffered preservative solution (e.g., methanol-water-based). In a specific embodiment, the container contains ThinPrep® solution.

In various embodiments, the kit includes at least one primer or probe whose binding distinguishes between a methylated and an unmethylated sequence, together with instructions for using the primer or probe to identify a neoplasia. In another embodiment, the kit further comprises a pair of primers suitable for use in a polymerase chain reaction (PCR). In yet another embodiment, the kit further comprises a detectable probe. In yet another embodiment, the kit further comprises a pair of primers capable of binding to and amplifying a reference sequence.

In yet other embodiments, the kit comprises a sterile container which contains the primer or probe; such containers can be boxes, ampules, bottles, vials, tubes, bags, pouches, blister-packs, or other suitable container form known in the art. Such containers can be made of plastic, glass, laminated paper, metal foil, or other materials suitable for holding nucleic acids. The instructions will generally include information about the use of the primers or probes described herein and their use in diagnosing a neoplasia. Preferably, the kit further comprises any one or more of the reagents described in the diagnostic assays described herein. In other embodiments, the instructions include at least one of the following: description of the primer or probe; methods for using the enclosed materials for the diagnosis of a neoplasia; precautions; warnings; indications; clinical or research studies; and/or references. The instructions may be printed directly on the container (when present), or as a label applied to the container, or as a separate sheet, pamphlet, card, or folder supplied in or with the container.

### VII. Algorithm for Predicting ESCC

As described herein, in certain embodiments, the methods of the present invention can utilize least absolute shrinkage and selection operator technique (Lasso) with linear regression.

In other embodiments, any number of algorithms that can capture linear effects (e.g., linear regression) or both linear and non-linear effects (e.g., Random Forest, Gradient Boosting, Neural Networks (e.g., deep neural network, extreme learning machine (ELM)), Support Vector Machine, Hidden Markov model) can be used in the methods described herein. See, for example, McKinney et al., 2011, Appl. Bioinform., 5(2):77-88; Gunther et al., 2012, BMC Genet., 13:37; and Ogutu et al., 2011, BMC Proceedings, 5(Suppl 3):Sl 1. Any type of machine learning algorithm or deep learning neural network algorithm (tuned or non-tuned) capable of capturing linear and/or non-linear contribution of traits for the prediction can be used. In some instances, a combination of algorithms (e.g., a combination or ensemble of multiple algorithms that capture linear and/or non-linear contributions of traits) is used.

Simply by way of example, Random Forest™ is a popular machine learning algorithm created by Breiman & Cutler for generating “classification trees” (see, for example, “stat.berkeley.edu/breiman/RandomForests/cc_home.htm” on the World Wide Web). Using standard machine learning and predictive modeling techniques, a diagnostic classifier algorithm was written to be implemented in R and Python programming languages (though it can be implemented in many other programming languages), according to well described guidelines by Breiman & Cutler. A diagnostic classifier algorithm was generated using data from at least two traits (T) and the diagnosis of interest from that population. To determine the output (e.g., diagnosis) for a new individual, one simply determines values for the at least two traits (T) and inputs that information into an algorithm (e.g., the diagnostic classifier algorithm described herein or another algorithm discussed above) that is capable of capturing the linear and non-linear contributions of the traits.

As described herein, the inputs are the methylation status of at least one CpG dinucleotide, and the outcome can represent a positive or a negative probability (e.g., prediction or diagnosis) for ESCC. The Traits (T) used to determine the outcome can represent the methylation status of at least one CpG dinucleotide, but Traits (T) also can correspond to at least one interaction (e.g., between the methylation status of two different sites (CpG×CpG)). It would be appreciated that any such interactions can be visualized using partial dependence plots.

It will be apparent that the present invention provides a skilled artisan the ability to construct a matrix in which the methylation status of one or more CpG dinucleotides can be evaluated as described herein, typically using a computer, to identify interactions and allow for prediction of ESCC. Although such an analysis is complex, no undue experimentation is required as all necessary information is either readily available to the skilled artisan or can be acquired by experimentation as described herein.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

Esophageal squamous cell carcinoma (ESCC) is the most common type of esophageal cancer worldwide, especially in certain geographic areas, such as China and Eastern Africa. It carries a very poor prognosis due to late diagnosis. Endoscopy with biopsy remains the gold standard for diagnosis. However, patients often only undergo endoscopy after tumor growth has caused symptoms, such as difficulty swallowing. Therefore, a nonendoscopic method of screening and early detection would vastly improve outcome, especially in developing countries with limited resources, where more than 80% of ESCC deaths occur. Biomarker-based prediction panels, particularly those employing minimally invasive sampling techniques, will be highly useful in detecting ESCC early, as well as in stratifying high- vs. low-risk patients for more efficient follow-up endoscopy or future treatment regimens.

Currently, there are no established population-based screening modalities for ESCC. An obvious recourse has been to identify molecular features, possibly as adjuncts to histology, to better diagnose and risk-stratify patients. However, this recourse still requires endoscopy as a first line of detection. Esophageal cytology is an easy and inexpensive sampling technique, but current methods are insufficient for primary screening due to poor sensitivity. Since aberrant DNA methylation of tumor suppressor genes occurs in ESCC, with higher methylation levels in tumor tissues than in normal esophagus, we devised a strategy to combine non-invasive sampling techniques with novel methylation biomarkers for early diagnosis and risk prediction in ESCC and its precursor dysplastic lesions.

One non-invasive, inexpensive sampling technique is the EsophaCap sponge capsule, a string-attached, gelatin-enclosed sponge that dissolves when swallowed. The sponge then expands and is retrieved via the string, collecting esophageal cells as it exits. These cells can then be tested with a panel of methylation probes to measure methylation levels, diagnose ESCC or esophageal dysplasia, and stratify high- vs. low-risk patients.

We have identified five novel markers which are able to detect significant methylation abnormalities in ESCC versus non-cancerous esophagus: (1) cg20655070; (2) TAC1; (3) ZNF542; (4) SLC35F1; and (5) ZNF132. These novel biomarkers can be used on any collection of esophageal cells, e.g., obtained via non-invasive sampling techniques such as the EsophaCap device, for ESCC risk stratification. This strategy is especially beneficial for high-risk patients in low-income areas with limited access to endoscopy and other medical resources.

### Example 1: A Gene Methylation Assay that can Accurately Distinguish Between Normal Esophageal and ESCC Tissue Samples

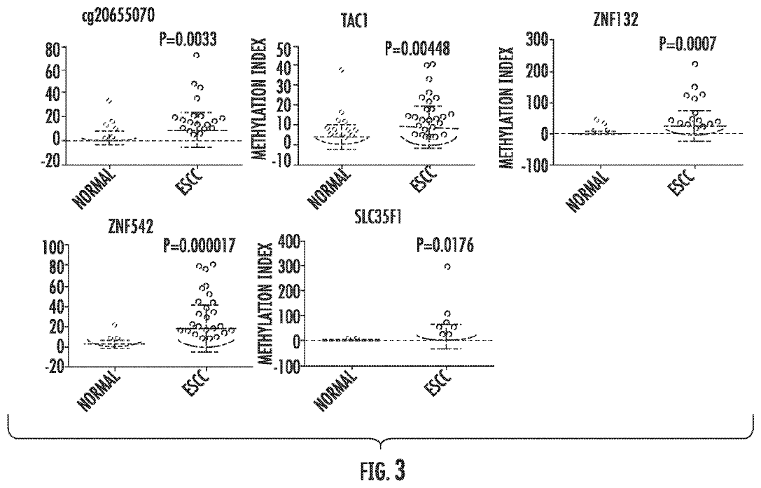

The experimental design of the ESCC study began with biomarker selection from publications and the DNA methylation microarrays of The Cancer Genome Atlas (TCGA) database. There were 93 ESCC samples in TCGA along with 14 normal adjacent esophageal samples. To increase the sample size for more robust biomarker search, we also included 120 normal samples across 12 different tissue types in our analysis. Using TCGA, we looked for CpG islands with at least 30% methylation in 50% or more of tumors and less than 5% methylation in normal tissues. Candidate regions with at least two eligible CpG sites were selected. From both literature review and TCGA, 32 regions were chosen for preliminary screening. After primer design and PCR testing with fully methylated and unmethylated DNA, 15 were picked for probe design and quantitative methylation-specific PCR (qMSP). Using a subset of ESCC and normal esophageal tissue DNAs for screening, the top six candidate regions were selected for further testing. ESCC and normal esophageal tissues were then obtained from 48 patients and DNA was extracted. After quantitation and verification of the absence of DNA degradation, the methylation-on-beads (MOB) method was used to measure methylation levels of the 5 final selected genes. As shown in FIG. 3 methylation levels of all 5 genes tested differed significantly between 48 matched ESCC and normal esophageal tissues.

### Example 2: Test the EsoCAN Panel on a Limited Series of Normal and ESCC Samples Obtained Via EsophaCap™ Sponge

The results shown in FIG. 3 under Example 1 demonstrated significantly higher methylation levels of five genes in ESCC vs. normal esophageal tissues. In this Example, the present invention investigated whether these markers could be used as a diagnostic test with the much more limited and less neoplastically pure DNA collected via EsophaCap. It should be emphasized that the EsophaCap™ collects cells along the entire length of the esophagus, not merely from the tumor; thus, substantial dilution of tumor DNA by normal esophageal cellular DNA occurs. Nevertheless, the present inventors' previous success with the EsoBEE assay to detect BE, which affects an even smaller portion of the esophagus than ESCC, provided confidence that an analogous panel would succeed in ESCC detection.

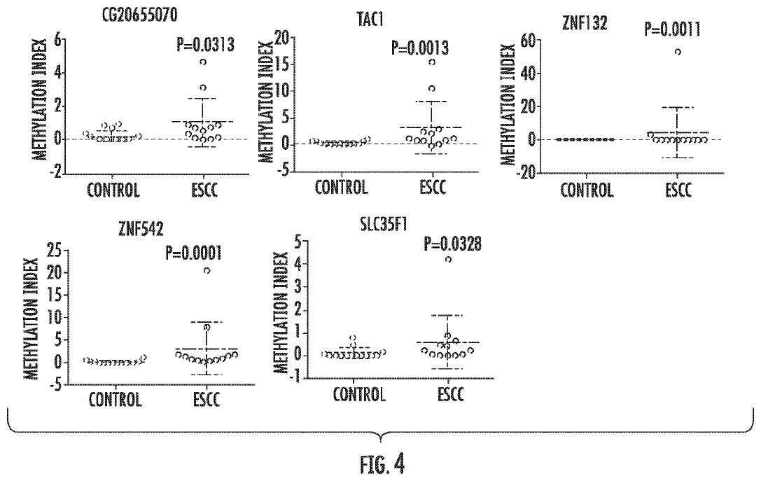

The diagnostic performance of our 5-gene tissue methylation biomarker panel was evaluated in EsophaCap™ samples from patients with ESCC vs. non-neoplastic controls. Thus far, samples from 26 participants have been collected (12 ESCC+14 non-neoplastic controls). As shown in FIG. 4, all 5 genes tested were accurate in distinguishing ESCC from non-cancer control patient EsophaCap™ samples.

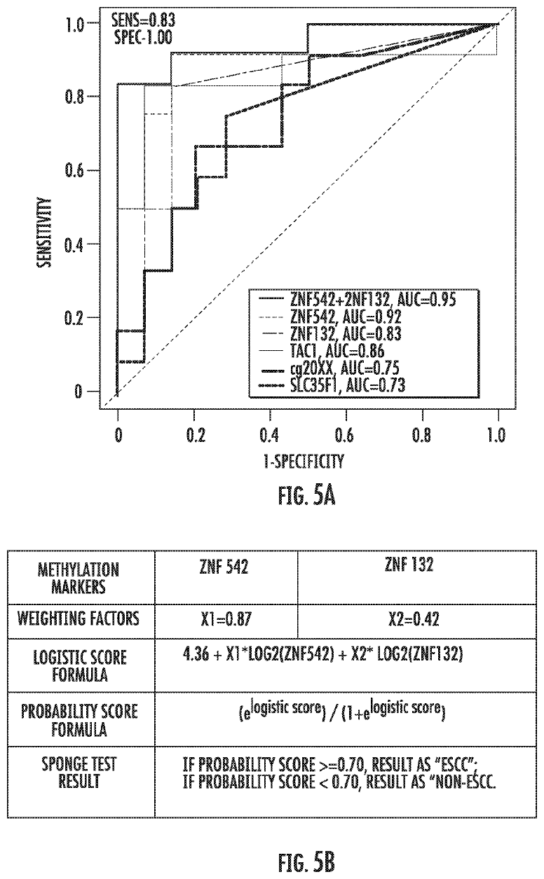

Combined statistical analyses of these 5 genes was performed to develop a multi-gene model to further optimize diagnostic accuracy. Univariate analysis was first conducted to evaluate each gene based on marker data from the 26 sponge participants. The receiver-operating characteristic (ROC) curve and the corresponding area under the curve (AUC) of each methylation marker in distinguishing ESCC are shown in FIG. 5A. Then, a multivariable model was constructed for combinations of genes, and a cut-off threshold was determined to classify disease status (ESCC vs. control). Specifically, least absolute shrinkage and selection operator technique (Lasso) was used to select the best markers among the five based on a logistic regression model. The model produces a score presenting the probability of ESCC based on a weighted sum of markers, in which the weights are the corresponding estimated coefficients from the logistic regression model. The ultimate goal is to develop this as a screening test for ESCC in high-incidence populations in resource-constrained settings where EGD is not routinely available. For a screening test to have good clinical utility, its specificity needs to be very high to yield a high negative predictive value and a reasonable positive predictive value. Therefore, the cut-off threshold of the score to define ESCC is chosen by maximizing sensitivity while constraining specificity to be ≥0.999. In one embodiment, the test comprises two markers: ZNF542 and ZNF132. The area under the receiver-operating characteristic (ROC) curve (AUC) based on this model in the training data is 0.95 (FIG. 5A). In this context, it should be noted that AUCs>0.750 constitute excellent biomarker performance, while AUCs>0.900 are considered outstanding (76-78). FIG. 8B shows each marker's weight in the model and calculation of the probability score. The score cut-off value to define ESCC is 0.70 (FIG. 5B). This cut-off threshold yields a sensitivity and specificity of 0.83 and 1.00, respectively, with a misclassification rate of only 7.7%. Thus, these findings strongly support the 2-marker embodiment and the chosen cut-off threshold to the pilot test-set data.

### Example 3: Analytically Validate a PCR-Based Assay for Detecting ESCC

The EsoCAN biomarker panel was developed by testing 48 matched normal and ESCC tissue biopsy samples, then by further testing on 12 ESCC and 14 non-neoplastic sponge samples. In this study, additional experiments are carried out to optimize and analytically validate the assay, as well as to validate the diagnostic accuracy of the top two candidate genes in the EsoCAN panel. Finally, concordance in assay results are established between sponges and their matched endoscopic biopsy counterparts.

Reproducibility: Technical Replicates.

First, 20 normal control and 20 ESCC patients will have three equal aliquots created from their sponge samples. DNA is extracted from each EsophaCap™ sponge aliquot and assayed for genes in the EsoCAN methylation panel. In addition, ten individual tissue samples are divided into thirds, and DNA is extracted from each aliquot and evaluated by the EsoCAN panel.

Protocol: DNA Extraction from Tissue Biopsy Samples—Methylation on Beads (MOB).

DNA Extraction from Tissues:

After the biopsy is obtained during EGD, it is immediately frozen on dry ice, and stored in a −80° C. freezer or in a liquid nitrogen tank. DNA is extracted using the Qiagen Tissue Kit (Cat. #69504, Germantown, Md.).

Methylation on Beads (MOB):

Briefly, 1 ug of genomic DNA in 20 ul is mixed with 50 ul of magnetic beads, and CT Conversion Reagent is added and the sample is heated to 98° C. for 8 minutes, followed by heating at 55° C. for 60 minutes. After a series of washes using a magnetic stand, L-Desulphonation Buffer is added and the beads are incubated at room temperature for 13 minutes. After another set of exhaustive wash cycles, the DNA is eluted with Elution Buffer and stored. This procedure is performed as described by us for small samples (72, 73, 79).

Quantitative Methylation-Specific PCR (qMSP) Assays:

These are performed as previously described; notably, these experiments have been optimized in Dr. Meltzer's laboratory at Johns Hopkins University. Before a specific gene can be considered for inclusion in the development of a methylation-specific assay for the detection of ESCC, several different combinations of possible primer pairs must undergo many rounds of testing using a variety of annealing temperatures and other conditions.

Statistical Considerations:

The analytical performance of the assay is evaluated and reported using metrics including as accuracy, within-run and between-run precision, analytical and functional sensitivity, linearity, as well as reference range determination. Repeatability among technical replicates is quantified with coefficient of variation, as well as intra-class correlation coefficients (ICC) using variance component analysis from repeated measures of the samples in different batches, equipment, and days.

For concordance between EsophaCap™-based analyses and matching tissue biopsy samples, a newly collected set of 20 ESCC case and 20 control sponge samples in parallel are analyzed with their matched endoscopic biopsy samples. Concordance of marker detection is determined between sponge and tissue using the kappa statistic. A kappa of 0.5 or lower is considered insufficient, while the minimum required level for adequate concordance is a kappa of 0.85. A sample size of 40 pairs of tissue and sponge samples (20 from cases and 20 from controls) provides 84% power to detect a kappa of 0.85 rejecting the null value of 0.5, using a two-sided test with a significance level of 0.05.

High repeatability is expected and determined by repeat assays of aliquots from selected sponge samples. High analytical sensitivity (1:1,000 dilution of methylated DNA) and specificity (<5% methylation in unmethylated control DNA) is expected. It is further expected that repeatability meets a threshold of 90% in duplicate aliquots of 10 samples (20 aliquots). Concordance is expected to meet a threshold of kappa=0.85 in 40 matched sponge/endoscopic biopsy pairs (from 20 ESCC and 20 non-cancer control patients).

### Example 4: Conduct a Pilot Study of the EsoCAN Assay on Specimens Collected at JHU Via EsophaCap

EsoCAN is supported by strong preliminary data showing high diagnostic accuracy in ESCC tissues, as well as in a pilot study of EsophaCap™ sponge-collected specimens from ESCC patients and controls. This Example will 1) establish diagnostic accuracy of EsophaCap™/EsoCAN vs. EGD-obtained biopsy (the gold-standard method) and 2) perform independent assessments of interference among combined biomarker probes in detection accuracy. Lugol staining is also performed to verify absence of dysplasia in biopsies from endoscopic areas used as normal control tissues. Ninety percent diagnostic accuracy of EsoCAN in EsophaCaps is expected from 30 ESCC and 30 non-neoplastic control patients.

Based on the 14-normal, 12-ESCC sponge sample training set, the present inventors constructed a 2-marker prediction model for ESCC and chose a cut-off threshold to define ESCC based on test results. This model and the chosen cut-off threshold is applied to a set of 60 untested samples obtained using EsophaCaps, then the misclassification rate of the test is estimated. This test set comprises 30 ESCC patients and 30 non-ESCC controls.

The present inventors have built, in one embodiment, a 2-gene model that exhibits excellent diagnostic performance for ESCC based on an interval cross-validation procedure. Before initiating a large confirmative study, a validation study is conducted with an independent test set of patients to estimate the misclassification rate. It is expected that the model demonstrates high classification accuracy. If the model does not perform well in this independent set of patients, the training step is repeated using a new patient sample set, reasoning that the initial training cohort may have been skewed so the model cannot perform well in the new test set of patients. Alternatively, the training step is repeated using the pooled data from both the original patient cohort (12 cases/14 controls) and the new cohort, but applying different statistical strategies to create the model (e.g., classification tree, artificial neural networks, or support vector machines).

Protocol: Patient Recruitment. Patient Cohort:

Any patient who is undergoing endoscopy for any reason qualifies for this study. The JHU endoscopy practice, which performs at least 100 EGDs per week, is used to prospectively obtain sponge samples and tissue specimens, including all patients enrolled in this study.

Pathological Evaluation.

All ESCC cases and controls are validated by corresponding EGD sampling and histologic assessment by a qualified GI pathologist.

Sponge Administration.

EsophaCap™ sponges are available in various diameters, ranging from 10 mm to 35 mm. In particular embodiments, the optimal diameter for obtaining adequate esophageal samples while maintaining patient comfort is 20 mm (data not shown). The plastic foam expands when the gelatin capsule dissolves; the pore size (roughness) of this foam can also be adjusted from 10 to 50 units, and a pore size of 20 mm maintains abrasion while minimizing undue mucosal trauma (47, 48). The sponge is administered immediately prior to endoscopy by the clinical coordinator in clinic or the endoscopy suite, while the patient is conscious. After 3 min, the capsule dissolves and the sponge is withdrawn via the external oral string. EsophaCap™ is FDA 510k-cleared and CE-marked, and JHU Institutional Review Board (IRB) approval has been obtained for its use in patients. It can be administered in outpatient clinics, before endoscopy, or after endoscopy—yielding versatility and ease of patient enrollment.

Sponge Processing.

In particular embodiments, after the EsophaCap™ sponge is withdrawn from the patient, it is placed in 20 ml of ThinPrep solution, and shaken for three minutes. The sponge is washed two times with 20 ml of PBS. Each solution that is collected is centrifuged at 2500 rpm for 10 minutes and the pellets are collected and pooled. Proteinase K (Cat. #P8107S, New England Biol.) is added and after an overnight digestion the DNA is extracted using a DNeasy Kit (Qiagen).

MOB and qMSP Assays.

These assays are performed as described above.

Statistical Considerations.

Subjects are classified as either ‘ESCC’ or ‘non-ESCC’ based on rules developed from the training data. The fraction correctly classified (true ESCC positive+true negative) is determined, whereas misclassification rate equals the fraction classified incorrectly.

Sample Size Determination.

It is expected that the misclassification rate of the model will be 10% according to preliminary data; a sample size of 60 provides 85% power to reject the null misclassification rate of 25% in favor of 10%, using a two-sided binomial exact test with a significance level of 0.05.

### Example 5: Clinically Validate the EsoCAN Assay in a Case-Control Study in Tanzania

The sensitivity and specificity of the EsoCAN biomarker panel in EsophaCap™ sponge samples is evaluated in 300 patients in Tanzania, including 75 cases with confirmed ESCC and 225 controls. The samples are stored in ThinPrep solution at room temperature, shipped to the United States, and analyzed in a laboratory. The methylation detection device is analytically validated using samples from confirmed cases of ESCC at Muhimbili National Hospital in Tanzania. The methylation analysis for two methylation biomarkers (ZNF132 and ZNF542), which will be normalized to beta-actin, is performed. Agreement by +/−10% between sets of normalized methylation values (NMVs) is taken as evidence of analytical validation.

Prior to inception of the study and IRB submissions, informational sessions are conducted with frontline staff at Muhimbili National Hospital in Tanzania for feedback on the device and the collection protocol. These sessions provide a background on the development of the device, a hands-on instructional session about how to use it, and an overview of the protocol with detailed time for feedback on how to modify the protocol for the environment. The protocol is modified as needed to ensure that the sponge is usable not only in a trial setting, but in a real-world setting in East Africa. The final protocol is submitted for approval by the institutional review boards at Muhimbili University of Health and Allied Sciences, the University of California, San Francisco, and Johns Hopkins University.

Cases are matched to controls by gender and age+/−10 years. Based upon previous reports, it is expected that cases are 2:1 male to female, and approximately 20% of cases are <40(3). Regarding stage, it is expected that most cases are very advanced at the time of presentation; therefore, the sample is not restricted to include a significant number of early cases. Additionally, staging imaging in Tanzania is limited; thus, the limitations of reporting accurate staging for many patients in this setting is acknowledged. Relevant clinical data is abstracted, including any diagnostic imaging performed, but no analyses are recruited or stratified according to this variable, since it is somewhat unreliable.

Study staff utilize previously developed rapid ascertainment methods to identify patients with a suspected diagnosis of ESCC from the surgical wards and outpatient endoscopy unit at MNH. Patients consent for biospecimen collection prior to the procedure; however, in some cases consent is obtained following diagnosis. Because costs of pathologic confirmation are often prohibitive to patients in Tanzania, fees are waived for study participants as compensation for participation. All specimens undergo formal pathologic review for confirmation of ESCC at the Central Pathology Lab at Muhimbili National Hospital, and only specimens with a confirmed diagnosis of ESCC are included in the analysis detailed below.

Controls are recruited from patients hospitalized on the medical and surgical wards at Muhimbili National Hospital for non-malignant conditions. Patients with symptoms of dysphagia severe enough to prevent swallowing of the capsule are excluded; in the present inventors' experience, patients with mild or moderate dysphagia can still swallow the capsule.

Any health assistant can perform sponge administration with minimal training. The sponge is swallowed on-site, under the supervision of a research assistant, with retrieval about 5 minutes post-deglutition, either immediately preceding or following EGD in the Endoscopy Unit at Muhimbili National Hospital. As an added precaution, the sponge contains a small radiopaque nontoxic disc tracer so that it can be located by X-ray, in the unlikely event that it becomes severed or otherwise irretrievable via the monofilament line. Further safety is conferred by the attachment of the device to a firm 30-lb.-weight monofilament (fishing) line, which is stronger and more durable than other materials used previously in similar devices (49, 50).

Cases and controls are frequency-matched by age and gender (2:1 male:female; see above). Age- and sex-adjusted sensitivity and specificity are estimated to characterize the performance of the assay using a stratified analysis.

It is stipulated that the minimum acceptable sensitivity and specificity of the assay to detect ESCC are 45% and 97%, respectively (the null hypothesis). However, based on preliminary results, it is expected that the test performs even better—viz., at 65% sensitivity and 99.8% specificity (the alternative hypothesis). A sample size of 75 ESCC cases and 225 controls provides 90% power to reject the null hypothesis of 45% sensitivity and 97% specificity in favor of the alternative hypothesis of 65% sensitivity and 99.8% specificity with the present assay, based on their joint 95% confidence region. With the alternative hypothesis values and a 0.08% population prevalence for ESCC, the corresponding PPV and NPV are 0.206 and 1.000, respectively. That is, at least 206 ESCCs will be diagnosed in every 1000 patients undergoing endoscopy subsequent to a positive test result, while no one with a negative result will have ESCC, so the test will not miss the diagnosis of any true ESCC. These PPV and NPV values are reasonable and acceptable in the context of an eventual screening test, particularly where no alternative screening test currently exists.

### Example 6: Assay Protocols

DNA Extraction from Sponge:

1. Vigorously shake for 2-3 minutes ThinPrep container with sponge.

2. Transfer all solution from ThinPrep container to new 50 ml tube.

3. Centrifuge for 10 min at 2500 RPM.

4. Remove and discard supernatant.

5. Add 25 mL PBS to original ThinPrep container and vigorously shake for 2-3 minutes. Transfer solution to previous 50 mL tube (from step 2 containing the pellet).

6. Centrifuge for 10 min at 2500 RPM.

7. Remove and discard supernatant, making sure not to disturb pellet.

8. Add 3 ml PBS and suspend pellet. Transfer solution to two 2 mL tubes.

9. Centrifuge for 5 min at 2500 RPM.

10. Remove and discard supernatant, making sure not to disturb pellet. Can proceed directly to DNA extraction or pellet can be stored at −80° C.

11. Add 1100 ul ATL+100 ul proteinase K (NEB P8107S) to the cell pellet and vortex.

12. Shake (40 rpm) in 56° C. water bath overnight.

13. Next day morning, add 20 uL proteinase K and vortex. Place back into 56° C. water bath for an additional 2 hours.

14. Follow the Dneasy kit (QIAGEN) for finale steps in DNA extraction (details below).

15. Add 2.4 mL of Al buffer and ethanol mix (in 1:1 ratio) to 15 ml tubes, transfer entire cell lysis from step 13 to 15 mL tube and vortex.

16. Transfer 700 uL solution from step 15 to Qiagen spin column. Centrifuge at 9000 rpm for 1 min and discard flow-through. Repeat this step as needed until no more DNA solution remains in 15 ml tube.

17. Wash spin column with 500 uL Wash Buffer 1. Discard flow-through.

18. Wash spin column with 500 uL Wash Buffer 2. Discard flow-through.

19. Centrifuge spin column for 3 min at 14000 rpm.

20. Place spin column in 1.5 ml tube. Add 50 uL water to column, incubate for 1 min, and then centrifuge for 1 min at 9000 rpm.

21. Repeat step 20 for additional aliquot of extracted DNA.

### Bisulfite Treatment Procedure:

1. Add 1 ug DNA (+H2O for total of 20 uL) into 1.5 ml tube.

2. Add 50 uL of magnetic beads to sample tube. Mix DNA and beads on the rocker for 10 minutes.

3. Add 130 μl of prepared lightning CT conversion reagent to each sample tube

4. Incubate at 98° C. for 8 minutes followed by 55° C. for 60 minutes.

5. Place the tubes on ice for 10 minutes.

6. Add 400 μl of M-Binding Buffer.

7. Incubate at room temperature for 5 minutes.

8. Add 2 μl of carrier RNA.

9. Place the tube on a magnetic holder and discard the supernatant.

10. Remove the tube from the magnetic holder, add 400 μl of M-Wash Buffer, and mix.

11. Place the tube on the magnetic holder and discard the supernatant. Centrifuge the tubes and place in the magnetic holder again to remove as much liquid as possible.

12. Add 200 μl of L-Desulphonation Buffer and mix.

13. Incubate at room temperature for 13 minutes.

14. Add 2 μl of carrier RNA and incubate at room temperature for 2 minutes.

15. Place the tube on the magnetic holder and remove the supernatant.

16. Add 400 μl of M-Wash Buffer and mix.

17. Place the tube on the magnetic holder and remove the supernatant.

18. Repeat the previous two wash steps one more time. Discard supernatant completely after this washing step, leaving only the magnetic particles with DNA in the tube. Centrifuge the tubes and place in the magnetic holder again to remove as much liquid as possible.

19. All ethanol must be removed. Air-dry with the cap open at 90° C. for 10 minutes.

20. Elute the DNA from the magnetic beads by adding 50 μl of M-Elution/(DNA elution) buffer to the magnetic beads. Incubate at 90° C. for 10 min.

21. Place in magnetic rack and transfer liquid to a new tube. Do not discard the tube with beads.

22. Add another 50 μl of M-Elution/(DNA elution) buffer to the magnetic beads. Incubate at 90° C. for 10 min.

23. Place the tube containing the beads in the magnetic holder and transfer the liquid to the tube containing the initial 50 μl. The final volume in this tube should be close to 100 μl. The tube with the beads may now be discarded.

qMSP:

1. Stage 1
a. 95° C. for 5 min.

2. Stage 2. Repeat 40 times.
a. 95° C. for 15 sec
b. 60° C. for 25 sec
c. 72° C. for 30 sec

TABLE 1
qMSP Primers and Probes

| Gene | MSP Primer | 5′->3′ |
|---|---|---|
| ZNF542 | F R Probe | TAGGTTTCGCGTCGAGGTTTTAC (SEQ ID NO: 1) ACGACCCCGCTCCCAATAACCGAA (SEQ ID NO: 2) CGTACGCGTATTTTGTGTTTTAGG (SEQ ID NO: 3) |
| ZNF132 | F R Probe | GTAGTAAAATGAGGATCGTAATGGC (SEQ ID NO: 4) CGTAACGAACATAAAAAAATAACGTC (SEQ ID NO: 5) TAGTTCGGATTTGTTATTGGTTCG (SEQ ID NO: 6) |
| cg20655070 | F R Probe | CGTCGTTTCGGGAGAGAGTGTC (SEQ ID NO: 7) CGCAAACCGAACCAAACACAACG (SEQ ID NO: 8) CGGGTAGTAGACGTCGAGGTTT (SEQ ID NO: 9) |
| TAC1 | F R Probe | GGGTATCGACGAGTTATCGTTTC (SEQ ID NO: 10) CGCGTGGGGAGAATGTTACG (SEQ ID NO: 11) CGTAAGCGAAAGGAGAGGAGGCG (SEQ ID NO: 12) |
| SLC35F1 | F R Probe | CGTAGTAGTAGTTGTAGTCGTCGTC (SEQ ID NO: 13) AACACTTTACGAATCCTCTAACG (SEQ ID NO: 14) ATTATTATCGAGAATTTGTCGGTCG (SEQ ID NO: 15) |

### REFERENCES

1. Murphy G, McCormack V, Abedi-Ardekani B, Arnold M, Camargo M C, Dar N A, Dawsey S M, Etemadi A, Fitzgerald R C, Fleischer D E, Freedman N D, Goldstein A M, Gopal S, Hashemian M, Hu N, Hyland P L, Kaimila B, Kamangar F, Malekzadeh R, Mathew C G, Menya D, Mulima G, Mwachiro M M, Mwasamwaja A, Pritchett N, Qiao Y L, Ribeiro-Pinto L F, Ricciardone M, Schuz J, Sitas F, Taylor P R, Van Loon K, Wang S M, Wei W Q, Wild C P, Wu C, Abnet C C, Chanock S J, Brennan P. International cancer seminars: a focus on esophageal squamous cell carcinoma. Annals of oncology: official journal of the European Society for Medical Oncology/ESMO. 2017; 28(9):2086-93. Epub 2017 Sep. 16. doi: 10.1093/annonc/mdx279. PubMed PMID: 28911061.
2. Van Loon K, Mwachiro M M, Abnet C C, Akoko L, Assefa M, Burgert S L, Chasimpha S, Dzamalala C, Fleischer D E, Gopal S, Iyer P G, Kaimila B, Kayamba V, Kelly P, Leon M E, Mathew C G, Menya D, Middleton D, Mlombe Y, Mmbaga B T, Mmbaga E, Mulima G, Murphy G, Mushi B, Mwanga A, Mwasamwaja A, Parker M I, Pritchett N, Schuz J, Topazian M D, White R E, McCormack V, Dawsey S M. The African Esophageal Cancer Consortium: A Call to Action. J Glob Oncol. 2018; 4:1-9. Epub 2018 Sep. 23. doi: 10.1200/JGO.17.00163. PubMed PMID: 30241229; PMCID: PMC6223465.
3. Mmbaga E J, Deardorff K V, Mushi B, Mgisha W, Merritt M, Hiatt R A, Mwaiselage J, Zhang L, Van Loon K. Characteristics of Esophageal Cancer Cases in Tanzania. J Glob Oncol. 2018; 4:1-10. Epub 2018 Sep. 23. doi: 10.1200/JGO.2016.006619. PubMed PMID: 30241222; PMCID: PMC6180793.
4. Jemal A, Bray F, Forman D, O'Brien M, Ferlay J, Center M, Parkin D M. Cancer burden in Africa and opportunities for prevention. Cancer. 2012; 118(18):4372-84. Epub 2012 Jan. 19. doi: 10.1002/cncr.27410. PubMed PMID: 22252462.
5. Cheng M L, Zhang L, Borok M, Chokunonga E, Dzamamala C, Korir A, Wabinga H R, Hiatt R A, Parkin D M, Van Loon K. The incidence of oesophageal cancer in Eastern Africa: identification of a new geographic hot spot? Cancer epidemiology. 2015; 39(2):143-9. Epub 2015 Feb. 11. doi: 10.1016/j.canep.2015.01.001. PubMed PMID: 25662402; PMCID: PMC4470609.
6. Sato F, Tsuchiya S, Meltzer S J, Shimizu K. MicroRNAs and Epigenetics. FEBS J. 2011; 278(10):1598-609. Epub 2011 Mar. 15. doi: 10.1111/j.1742-4658.2011.08089.x. PubMed PMID: 21395977.
7. Bouvier A M, Launoy G, Lepage C, Faivre J. Trends in the management and survival of digestive tract cancers among patients aged over 80 years. Aliment Pharmacol Ther. 2005; 22(3):233-41. doi: 10.1111/j.1365-2036.2005.02559.x. PubMed PMID: 16091061.
8. Launay L, Dejardin O, Pornet C, Morlais F, Guittet L, Launoy G, Bouvier V. Influence of socioeconomic environment on survival in patients diagnosed with esophageal cancer: a population-based study. Diseases of the esophagus: official journal of the International Society for Diseases of the Esophagus/ISDE. 2012; 25(8):723-30. Epub 2012 Feb. 2. doi: 10.1111/j.1442-2050.2011.01312.x. PubMed PMID: 22292704.
9. Wei W Q, Chen Z F, He Y T, Feng H, Hou J, Lin D M, Li X Q, Guo C L, Li S S, Wang G Q, Dong Z W, Abnet C C, Qiao Y L. Long-Term Follow-Up of a Community Assignment, One-Time Endoscopic Screening Study of Esophageal Cancer in China. Journal of clinical oncology: official journal of the American Society of Clinical Oncology. 2015; 33(17):1951-7. doi: 10.1200/JCO.2014.58.0423. PubMed PMID: 25940715.
10. Kastelein F, van Olphen S H, Steyerberg E W, Spaander M C, Bruno M J. Impact of surveillance for Barrett's oesophagus on tumour stage and survival of patients with neoplastic progression. Gut. 2016; 65(4):548-54. Epub 2015 Apr. 24. doi: 10.1136/gutjnl-2014-308802. PubMed PMID: 25903690.
11. Lightdale C J, Winawer S J. Screening diagnosis and staging of esophageal cancer. Seminars in oncology. 1984; 11(2):101-12. PubMed PMID: 6729490.
12. Greenebaum E, Schreiber K, Shu Y J, Koss L G. Use of the esophageal balloon in the diagnosis of carcinomas of the head, neck and upper gastrointestinal tract. Acta cytologica. 1984; 28(1):9-15. PubMed PMID: 6582746.
13. Huang G J. Early detection and surgical treatment of esophageal carcinoma. Jpn J Surg. 1981; 11(6):399-405. PubMed PMID: 7328933.
14. Berry A V, Baskind A F, Hamilton D G. Cytologic screening for esophageal cancer. Acta cytologica. 1981; 25(2):135-41. PubMed PMID: 6941611.
15. Shen O, Liu S F, Dawsey S M, Cao J, Zhou B, Wang D Y, Cao S G, Zhao H Z, Li G Y, Taylor P R, et al. Cytologic screening for esophageal cancer: results from 12,877 subjects from a high-risk population in China. Int J Cancer. 1993; 54(2):185-8. PubMed PMID: 8486421.
16. Dawsey S M, Yu Y, Taylor P R, Li J Y, Shen Q, Shu Y J, Liu S F, Zhao H Z, Cao S G, Wang G Q, et al. Esophageal cytology and subsequent risk of esophageal cancer. A prospective follow-up study from Linxian, China. Acta cytologica. 1994; 38(2):183-92. PubMed PMID: 8147209.
17. Liu S F, Shen Q, Dawsey S M, Wang G Q, Nieberg R K, Wang Z Y, Weiner M, Zhou B, Cao J, Yu Y, et al. Esophageal balloon cytology and subsequent risk of esophageal and gastric-cardia cancer in a high-risk Chinese population. Int J Cancer. 1994; 57(6):775-80. PubMed PMID: 8206671.
18. Roth M J, Liu S F, Dawsey S M, Zhou B, Copeland C, Wang G Q, Solomon D, Baker S G, Giffen C A, Taylor P R. Cytologic detection of esophageal squamous cell carcinoma and precursor lesions using balloon and sponge samplers in asymptomatic adults in Linxian, China. Cancer. 1997; 80(11):2047-59. Epub 1997 Dec. 10. doi: 10.1002/(SICI)1097-0142(19971201)80:11<2047::AID-CNCR3>3.0.C O; 2-U [pii]. PubMed PMID: 9392326.
19. Jin Z, Cheng Y, Gu W, Zheng Y, Sato F, Mori Y, Olaru A V, Paun B C, Yang J, Kan T, Ito T, Hamilton J P, Selaru F M, Agarwal R, David S, Abraham J M, Wolfsen H C, Wallace M B, Shaheen N J, Washington K, Wang J, Canto M I, Bhattacharyya A, Nelson M A, Wagner P D, Romero Y, Wang K K, Feng Z, Sampliner R E, Meltzer S J. A multicenter, double-blinded validation study of methylation biomarkers for progression prediction in Barrett's esophagus. Cancer research. 2009; 69(10):4112-5. Epub 2009 May 14. doi: 0008-5472.CAN-09-0028 [pii] 10.1158/0008-5472.CAN-09-0028. PubMed PMID: 19435894.
20. Jin Z, Mori Y, Hamilton J P, Olaru A, Sato F, Yang J, Ito T, Kan T, Agarwal R, Meltzer S J. Hypermethylation of the somatostatin promoter is a common, early event in human esophageal carcinogenesis. Cancer. 2008; 112(1):43-9. PubMed PMID: 17999418.
21. Jin Z, Hamilton J P, Yang J, Mori Y, Olaru A, Sato F, Ito T, Kan T, Cheng Y, Paun B, David S, Beer D G, Agarwal R, Abraham J M, Meltzer S J. Hypermethylation of the AKAP12 Promoter is a Biomarker of Barrett's-Associated Esophageal Neoplastic Progression. Cancer epidemiology, biomarkers & prevention: a publication of the American Association for Cancer Research, cosponsored by the American Society of Preventive Oncology. 2008; 17(1):111-7. PubMed PMID: 18199717.
22. Jin Z, Cheng Y, Olaru A, Kan T, Yang J, Paun B, Ito T, Hamilton J P, David S, Agarwal R, Selaru F M, Sato F, Abraham J M, Beer D G, Mori Y, Shimada Y, Meltzer S J. Promoter hypermethylation of CDH13 is a common, early event in human esophageal adenocarcinogenesis and correlates with clinical risk factors. Int J Cancer. 2008; 123(10):2331-6. PubMed PMID: 18729198.
23. Jin Z, Olaru A, Yang J, Sato F, Cheng Y, Kan T, Mori Y, Mantzur C, Paun B, Hamilton J P, Ito T, Wang S, David S, Agarwal R, Beer D G, Abraham J M, Meltzer S J. Hypermethylation of tachykinin-1 is a potential biomarker in human esophageal cancer. Clinical cancer research: an official journal of the American Association for Cancer Research. 2007; 13(21):6293-300. PubMed PMID: 17975140.
24. Jin Z, Mori Y, Yang J, Sato F, Ito T, Cheng Y, Paun B, Hamilton J P, Kan T, Olaru A, David S, Agarwal R, Abraham J M, Beer D, Montgomery E, Meltzer S J. Hypermethylation of the nel-like 1 gene is a common and early event and is associated with poor prognosis in early-stage esophageal adenocarcinoma. Oncogene. 2007; 26(43):6332-40. PubMed PMID: 17452981.
25. Schulmann K, Sterian A, Berki A, Yin J, Sato F, Xu Y, Olaru A, Wang S, Mori Y, Deacu E, Hamilton J, Kan T, Krasna M J, Beer D G, Pepe M S, Abraham J M, Feng Z, Schmiegel W, Greenwald B D, Meltzer S J. Inactivation of p16, RUNX3, and HPP1 occurs early in Barrett's-associated neoplastic progression and predicts progression risk. Oncogene. 2005; 24(25):4138-48. Epub 2005 Apr. 13. doi: 10.1038/sj.onc.1208598. PubMed PMID: 15824739.
26. Sato F, Jin Z, Schulmann K, Wang J, Greenwald B D, Ito T, Kan T, Hamilton J P, Yang J, Paun B, David S, Olaru A, Cheng Y, Mori Y, Abraham J M, Yfantis H G, Wu T T, Fredericksen M B, Wang K K, Canto M, Romero Y, Feng Z, Meltzer S J. Three-tiered risk stratification model to predict progression in Barrett's esophagus using epigenetic and clinical features. PloS one. 2008; 3(4):e1890. PubMed PMID: 18382671.
27. Kuester D, Dar A A, Moskaluk C C, Krueger S, Meyer F, Hartig R, Stolte M, Malfertheiner P, Lippert H, Roessner A, El-Rifai W, Schneider-Stock R. Early involvement of death-associated protein kinase promoter hypermethylation in the carcinogenesis of Barrett's esophageal adenocarcinoma and its association with clinical progression. Neoplasia. 2007; 9(3):236-45. PubMed PMID: 17401463.
28. Sato F, Meltzer S J. CpG island hypermethylation in progression of esophageal and gastric cancer. Cancer. 2006; 106(3):483-93. PubMed PMID: 16362978.
29. Hamilton J P, Sato F, Jin Z, Greenwald B D, Ito T, Mori Y, Paun B C, Kan T, Cheng Y, Wang S, Yang J, Abraham J M, Meltzer S J. Reprimo methylation is a potential biomarker of Barrett's-Associated esophageal neoplastic progression. Clinical cancer research: an official journal of the American Association for Cancer Research. 2006; 12(22):6637-42. PubMed PMID: 17121882.
30. Clement G, Braunschweig R, Pasquier N, Bosman F T, Benhattar J. Methylation of APC, TIMP3, and TERT: a new predictive marker to distinguish Barrett's oesophagus patients at risk for malignant transformation. The Journal of pathology. 2006; 208(1):100-7. Epub 2005 Nov. 10. doi: 10.1002/path.1884. PubMed PMID: 16278815.
31. Zou H, Osborn N K, Harrington J J, Klatt K K, Molina J R, Burgart L J, Ahlquist D A. Frequent methylation of eyes absent 4 gene in Barrett's esophagus and esophageal adenocarcinoma. Cancer epidemiology, biomarkers & prevention: a publication of the American Association for Cancer Research, cosponsored by the American Society of Preventive Oncology. 2005; 14(4):830-4. PubMed PMID: 15824152.
32. Zou H, Molina J R, Harrington J J, Osborn N K, Klatt K K, Romero Y, Burgart L J, Ahlquist D A. Aberrant methylation of secreted frizzled-related protein genes in esophageal adenocarcinoma and Barrett's esophagus. Int J Cancer. 2005; 116(4):584-91. PubMed PMID: 15825175.
33. McManus D T, Olaru A, Meltzer S J. Biomarkers of esophageal adenocarcinoma and Barrett's esophagus. Cancer research. 2004; 64(5):1561-9. PubMed PMID: 14996709.
34. Wong D J, Paulson T G, Prevo L J, Galipeau P C, Longton G, Blount P L, Reid B J. p16(INK4a) lesions are common, early abnormalities that undergo clonal expansion in Barrett's metaplastic epithelium. Cancer research. 2001; 61(22):8284-9. Epub 2001 Nov. 24. PubMed PMID: 11719461.
35. Eads C A, Lord R V, Wickramasinghe K, Long T I, Kurumboor S K, Bernstein L, Peters J H, DeMeester S R, DeMeester T R, Skinner K A, Laird P W. Epigenetic patterns in the progression of esophageal adenocarcinoma. Cancer research. 2001; 61(8):3410-8. PubMed PMID: 11309301.
36. Eads C A, Lord R V, Kurumboor S K, Wickramasinghe K, Skinner M L, Long T I, Peters J H, DeMeester T R, Danenberg K D, Danenberg P V, Laird P W, Skinner K A. Fields of aberrant CpG island hypermethylation in Barrett's esophagus and associated adenocarcinoma. Cancer research. 2000; 60(18):5021-6. Epub 2000 Oct. 4. PubMed PMID: 11016622.
37. Wong D J, Barrett M T, Stoger R, Emond M J, Reid B J. p16INK4a promoter is hypermethylated at a high frequency in esophageal adenocarcinomas. Cancer research. 1997; 57(13):2619-22. Epub 1997 Jul. 1. PubMed PMID: 9205067.
38. Paulson T G, Galipeau P C, Xu L, Kissel H D, Li X, Blount P L, Sanchez C A, Odze R D, Reid B J. p16 mutation spectrum in the premalignant condition Barrett's esophagus. PloS one. 2008; 3(11):e3809. Epub 2008 Dec. 2. doi: 10.1371/journal.pone.0003809. PubMed PMID: 19043591.
39. Bian Y S, Osterheld M C, Fontolliet C, Bosman F T, Benhattar J. p16 inactivation by methylation of the CDKN2A promoter occurs early during neoplastic progression in Barrett's esophagus. Gastroenterology. 2002; 122(4):1113-21. Epub 2002 Mar. 23. PubMed PMID: 11910361.
40. Keeley B, Stark A, Pisanic T R, Kwak R, Zhang Y, Wrangle J, Baylin S, Herman J, Ahuja N, Brock M V, Wang T H. Extraction and processing of circulating DNA from large sample volumes using methylation on beads for the detection of rare epigenetic events. Clin Chim Acta. 2013; 425:169-75. doi: DOI 10.1016/j.cca.2013.07.023. PubMed PMID: ISI:000328306100031.
41. Bailey V J, Zhang Y, Keeley B P, Yin C O, Pelosky K L, Brock M, Baylin S B, Herman J G, Wang T H. Single-Tube Analysis of DNA Methylation with Silica Superparamagnetic Beads. Clinical chemistry. 2010; 56(6):1022-5. PubMed PMID: ISI:000278145000024.
42. Bailey V J, Zhang Y, Keeley B P, Yin C, Pelosky K L, Brock M, Baylin S B, Herman J G, Wang T H. Single-tube analysis of DNA methylation with silica superparamagnetic beads. Clin Chem. 2010; 56(6):1022-5. PubMed PMID: 20360128.
43. Guzzetta A A, Pisanic Ii T R, Sharma P, Yi J M, Stark A, Wang T H, Ahuja N. The promise of methylation on beads for cancer detection and treatment. Expert review of molecular diagnostics. 2014; 14(7):845-52. doi: 10.1586/14737159.2014.943665. PubMed PMID: 25136840.
44. DeLong E R, DeLong D M, Clarke-Pearson D L. Comparing the areas under two or more correlated receiver operating characteristic curves: a nonparametric approach. Biometrics. 1988; 44(3):837-45. PubMed PMID: 3203132.
45. Pepe M S, Zheng Y, Jin Y, Huang Y, Parikh C R, Levy W C. Evaluating the ROC performance of markers for future events. Lifetime data analysis. 2008; 14(1):86-113. Epub 2007 Dec. 8. doi: 10.1007/s10985-007-9073-x. PubMed PMID: 18064569.
46. Pepe M, Longton G, Janes H. Estimation and Comparison of Receiver Operating Characteristic Curves. Stata J. 2009; 9(1):1. PubMed PMID: 20161343; PMCID: 2774909.
47. Wang Z, Kambhampati S, Cheng Y, Ma K, Simsek C, Tieu A H, Abraham J M, Liu X, Prasath V, Duncan M D, Stark A, Trick A, Tsai H L, Wang H, He Y, Khashab M A, Ngamruengphong S, Shin E J, Wang T H, Meltzer S J. Methylation Biomarker Panel Performance in EsophaCap Cytology Samples for Diagnosing Barrett's Esophagus: A Prospective Validation Study. Clinical cancer research: an official journal of the American Association for Cancer Research. 2019. Epub 2019 Jan. 24. doi: 10.1158/1078-0432.CCR-18-3696. PubMed PMID: 30670490.
48. Iyer P G, Taylor W R, Johnson M L, Lansing R L, Maixner K A, Yab T C, Simonson J A, Devens M E, Slettedahl S W, Mahoney D W, Berger C K, Foote P H, Smyrk T C, Wang K K, Wolfsen H C, Ahlquist D A. Highly Discriminant Methylated DNA Markers for the Non-endoscopic Detection of Barrett's Esophagus. Am J Gastroenterol. 2018; 113(8):1156-66. Epub 2018 Jun. 13. doi: 10.1038/s41395-018-0107-7. PubMed PMID: 29891853.
49. Kadri S R, Lao-Sirieix P, O'Donovan M, Debiram I, Das M, Blazeby J M, Emery J, Boussioutas A, Morris H, Walter F M, Pharoah P, Hardwick R H, Fitzgerald R C. Acceptability and accuracy of a non-endoscopic screening test for Barrett's oesophagus in primary care: cohort study. BMJ. 2010; 341:c4372. Epub 2010 Sep. 14. doi: 10.1136/bmj.c4372. PubMed PMID: 20833740; PMCID: 2938899.
50. Lao-Sirieix P, Boussioutas A, Kadri S R, O'Donovan M, Debiram I, Das M, Harihar L, Fitzgerald R C. Non-endoscopic screening biomarkers for Barrett's oesophagus: from microarray analysis to the clinic. Gut. 2009; 58(11):1451-9. Epub 2009 Aug. 5. doi: 10.1136/gut.2009.180281. PubMed PMID: 19651633.

1. A method for identifying a subject having esophageal squamous cell carcinoma (ESCC) comprising the steps of:
(a) extracting genomic DNA from a sample obtained from the subject;
(b) performing a conversion reaction on the genomic DNA in vitro to convert unmethylated cytosine to uracil by deamination; and
(c) detecting nucleic acid methylation of one or more genes in the converted genomic DNA, wherein detecting nucleic acid methylation identifies the subject as having 10 ESCC.

2. The method of claim 1, wherein the one or more genes comprise ZNF542, ZNF132, cg20655070, TAC1 and SLC35F1.

3. The method of claim 1, wherein the one or more genes comprise ZNF542 and
ZNF132.

4. The method of claim 3, wherein step (c) further comprises detecting the nucleic acid methylation of one or more of cg20655070, TAC1 and SLC35F1.

5. The method of claim 1, wherein the detecting step (c) comprises a polymerase chain reaction (PCR)-based technique.

6. The method of claim 5, wherein the PCR-based technique is quantitative methylation
specific PCR (QMSP).

7. The method of claim 1, wherein steps (a) and (b) are performed using methylation on beads technique.

8. The method of claim 1, wherein the sample is a cell sample.

9. The method of claim 8, wherein the cell sample is retrieved using a swallowable sponge device.

10. The method of claim 1, further comprising the step (d) of performing an endoscopy on the subject.

11. A method for treating a subject having ESCC comprising the steps of:
(a) extracting genomic DNA from a sample obtained from the subject;
(b) performing a conversion reaction on the genomic DNA in vitro to convert unmethylated cytosine to uracil by deamination;
(c) detecting nucleic acid methylation of one or more genes in the converted genomic DNA, wherein detecting nucleic acid methylation identifies the subject as having ESCC; and
(d) administering to the subject one or more treatment modalities appropriate for a subject having ESCC.

12. The method of claim 11, wherein the one or more treatment modalities comprises endoscopic resection, surgery, chemotherapy, radiotherapy or combinations thereof

13. The method of claim 12, wherein an endoscopy is performed prior to the treatment of step (d).

14. The method of claim 11, wherein the one or more genes comprise ZNF542, ZNF132, cg20655070, TAC1 and SLC35F1.

15-20. (canceled)

21. The method of claim 20, wherein the cell sample is retrieved using a swallowable sponge device.

22. A method comprising the steps of:
(a) extracting genomic DNA from a sample obtained from the subject;
(b) performing a conversion reaction on the genomic DNA in vitro to convert unmethylated cytosine to uracil by deamination; and
(c) detecting nucleic acid methylation of ZNF542 and ZNF132 in the converted genomic DNA.

23-28. (canceled)

29. The method of claim 22, further comprising the step (d) of performing an endoscopy on the subject.

30. A kit comprising:
(a) a primer complementary to a bisulfite-converted nucleic acid sequence comprising a CpG dinucleotide in the ZNF542 gene; and
(b) a primer complementary to a bisulfite-converted nucleic acid sequence comprising a CpG dinucleotide in the ZNF132 gene.

31. The kit of claim 30, further comprising one or more of:
(c) a primer complementary to a bisulfite-converted nucleic acid sequence comprising a CpG dinucleotide in the cg20655070 gene;
(d) a primer complementary to a bisulfite-converted nucleic acid sequence comprising a CpG dinucleotide in the TAC1 gene; and
(e) a primer complementary to a bisulfite-converted nucleic acid sequence comprising a CpG dinucleotide in the SLC35F1 gene.

32. The kit of claim 30, wherein (a) comprises one or more of SEQ ID NOS: 1-3; or (b) comprises one or more of SEQ ID NOS:4-6; or (c) comprises one or more of SEQ ID NOS:7-9; or (d) comprises one or more of SEQ ID NOS:10-12; or (e) comprises on or more of SEQ ID NOS:13-15.

33-40. (canceled)


SEQUENCE LISTING (25 sequences)

SEQ ID NO: 1 - DNA, 23 bp, Artificial Sequence
  ZNF542 forward primer
taggtttcgcgtcgaggttttac

SEQ ID NO: 2 - DNA, 24 bp, Artificial Sequence
  ZNF542 reverse primer
acgaccccgctcccaataaccgaa

SEQ ID NO: 3 - DNA, 24 bp, Artificial Sequence
  ZNF542 probe
acgaccccgctcccaataaccgaa

SEQ ID NO: 4 - DNA, 25 bp, Artificial Sequence
  ZNF132 forward primer
gtagtaaaatgaggatcgtaatggc

SEQ ID NO: 5 - DNA, 26 bp, Artificial Sequence
  ZNF132 reverse primer
cgtaacgaacataaaaaaataacgtc

SEQ ID NO: 6 - DNA, 24 bp, Artificial Sequence
  ZNF132 probe
tagttcggatttgttattggttcg

SEQ ID NO: 7 - DNA, 22 bp, Artificial Sequence
  cg20655070 forward primer
cgtcgtttcgggagagagtgtc

SEQ ID NO: 8 - DNA, 23 bp, Artificial Sequence
  cg20655070 reverse primer
cgcaaaccgaaccaaacacaacg

SEQ ID NO: 9 - DNA, 22 bp, Artificial Sequence
  cg20655070 probe
cgggtagtagacgtcgaggttt

SEQ ID NO: 10 - DNA, 23 bp, Artificial Sequence
  TAC1 forward primer
gggtatcgacgagttatcgtttc

SEQ ID NO: 11 - DNA, 20 bp, Artificial Sequence
  TAC1 reverse primer
cgcgtggggagaatgttacg

SEQ ID NO: 12 - DNA, 23 bp, Artificial Sequence
  TAC1 probe
cgtaagcgaaaggagaggaggcg

SEQ ID NO: 13 - DNA, 25 bp, Artificial Sequence
  SLC35F1 forward primer
cgtagtagtagttgtagtcgtcgtc

SEQ ID NO: 14 - DNA, 23 bp, Artificial Sequence
  SLC35F1 reverse primer
aacactttacgaatcctctaacg

SEQ ID NO: 15 - DNA, 25 bp, Artificial Sequence
  SLC35F1 probe
attattatcgagaatttgtcggtcg

SEQ ID NO: 16 - DNA, 578 bp, Homo sapiens
  misc_feature (1)..(578): ZNF542 original DNA sequence, chr1956879418- 56879995
cgcttgattctctgcgcaggcgcagcgttctcaacaccgccaacgggcacaaagccacgcccccacggccgaactcacgcctacgtactcagtcaccagcggaaccctctccaccaggctccgcgtcgaggccctacggggcgtcaatacgcacgcgtactttgtgtcccaggtagctcgtctcttagccatgtacgcctctgcattccggtcactgggagcggggccgtgggggctgcacatgcgcactgcgagtcctcgcctactcccagaaaccacccgggcgcatacgcggtgcggccgctgcttcaggtcggcgggctccagggagacctgggtgggcagcgtcgccgtttctcctttcttgggcagtatttttcccagcgccacgcggaggctgggccattatgaggtttgtgttgcgtcggggcggtccgtctcagaggcgctccggggaggagtgggcggctgaatggccagaggcccagggatccgttcaggtcagcgctggcgtccgggcctgagtttggaggtggcgggtgccttacaagaatgctcgcgtgatcagtgtgttggcg

SEQ ID NO: 17 - DNA, 578 bp, Homo sapiens
  misc_feature (1)..(578): ZNF542 DNA sequence after bisulfite conversion
  misc_feature (115)..(230): DNA sequence amplified by qMSP
cgtttgattttttgcgtaggcgtagcgtttttaatatcgttaacgggtataaagttacgtttttacggtcgaatttacgtttacgtatttagttattagcggaattttttttattaggtttcgcgtcgaggttttacggggcgttaatacgtacgcgtattttgtgttttaggtagttcgttttttagttatgtacgtttttgtatttcggttattgggagcggggtcgtgggggttgtatatgcgtattgcgagttttcgtttatttttagaaattattcgggcgtatacgcggtgcggtcgttgttttaggtcggcgggttttagggagatttgggtgggtagcgtcgtcgtttttttttttttgggtagtattttttttagcgttacgcggaggttgggttattatgaggtttgtgttgcgtcggggcggttcgttttagaggcgtttcggggaggagtgggcggttgaatggttagaggtttagggattcgtttaggttagcgttggcgttcgggtttgagtttggaggtggcgggtgttttataagaatgttcgcgtgattagtgtgttggcg

SEQ ID NO: 18 - DNA, 1036 bp, Homo sapiens
  misc_feature (1)..(1036): ZNF132 original DNA sequence, chr1958951215- 58952250
cgctgggccgaaccctcacacttccgctgggtgacggtcgcactcaggacctgtcttcccaaatgcaaaatgcgcgcaaggcctctgggcaccgcagggacgaaggctgggtatggagaccctggagacgcgtggcgctggctccactttcgggaacaccttggctcataaaagcagagtaattagccgggcactatggtgcgtgtgaaggcgcggtgacggtccctgcacccactcccgtgccctggtgtggctccagaggcctgctgtagcccaacccaccagcagcaaaatgaggaccgcaatggcggcgccggaagtcccgcctctcaatgacagctcggacttgccactggtccgagctttcattggctcaaggatccccgccgtctatgacgctatttccctatgcccgtcacgctactgctaggtcgttgccaaggtgattgaggaatggcgtttattgcgtcgctgctcaggcaacgcaaactacattatccagaaggaccctcgcggtgcctcagggctggccattggcagccgaggagacaggcacttccgggcggagtgtaagacgctggccaatcacagcctggcagcgggacttccgtcgtcgtcctcggaccatcactttggcatttctcgattttgtctgcttctgaagggaccgcgttgtcaggcgagggacggaatcttggaggctccctgggcccatggaaacaggagcgaggaaggcacgagagtcggggaagttccgccttcttgacatacaagcgcccccaccgcggcgaatatcgcccctgagcccagtgttcgccccccacaacaccgcaccccccttccccaccctgtgccttagataaagccggcccagtgggaccctcaggttgcaccgcgcggaggtgtccgcggctctcgggagtcgtcgcccttggcgttatgttggctttagggagcgcgaggccgaggccaagaaaagctacgcattgggaggtcttgacccagtgtgcgcccggctgtaggcatggaacgtgcg

SEQ ID NO: 19 - DNA, 1036 bp, Homo sapiens
  misc_feature (1)..(1036): ZNF132 DNA sequence after bisulfite conversion
  misc_feature (285)..(420): DNA sequence amplified by qMSP
cgttgggtcgaatttttatattttcgttgggtgacggtcgtatttaggatttgtttttttaaatgtaaaatgcgcgtaaggtttttgggtatcgtagggacgaaggttgggtatggagattttggagacgcgtggcgttggttttattttcgggaatattttggtttataaaagtagagtaattagtcgggtattatggtgcgtgtgaaggcgcggtgacggtttttgtatttattttcgtgttttggtgtggttttagaggtttgttgtagtttaatttattagtagtaaaatgaggatcgtaatggcggcgtcggaagtttcgttttttaatgatagttcggatttgttattggttcgagtttttattggtttaaggattttcgtcgtttatgacgttatttttttatgttcgttacgttattgttaggtcgttgttaaggtgattgaggaatggcgtttattgcgtcgttgtttaggtaacgtaaattatattatttagaaggattttcgcggtgttttagggttggttattggtagtcgaggagataggtattttcgggcggagtgtaagacgttggttaattatagtttggtagcgggattttcgtcgtcgttttcggattattattttggtatttttcgattttgtttgtttttgaagggatcgcgttgttaggcgagggacggaattttggaggttttttgggtttatggaaataggagcgaggaaggtacgagagtcggggaagtttcgtttttttgatatataagcgtttttatcgcggcgaatatcgtttttgagtttagtgttcgttttttataatatcgtattttttttttttattttgtgttttagataaagtcggtttagtgggatttttaggttgtatcgcgcggaggtgttcgcggttttcgggagtcgtcgtttttggcgttatgttggttttagggagcgcgaggtcgaggttaagaaaagttacgtattgggaggttttgatttagtgtgcgttcggttgtaggtatggaacgtgcg

SEQ ID NO: 20 - DNA, 618 bp, Homo sapiens
  misc_feature (1)..(618): cg20655070 original DNA sequence, chr1940314727-40315344
ctcattcatacagaattttcagtatgatcaccaacaccagatcgcctgcagtccatgttcacacaacaggctgtttctctctgcctttgcgcaaaccattccaccacaccgcccccgacccccaaaacatccttccgtcttcaggtctcagctcagacagctcctagtcttccgggatgatcttcctaccaaggtctcctcgggttcccaccgcgttccatgccgcctccattagtgaacatacataggtccgcaacggggtcttgcctgtctctcccactgagcggggcttcccgcaagccgggccaggcacagcggccagtgatgtccgcgtgctggggcctcttcaggcctcggcgtctactgcccgctctggaactgtgagcgacaaccggcactctctcccgggacggcgccgcacccaccttctgcacacctcggagacctcaacacctgcgccaaagccacccagtcacagccttctctccctctcctaaacaccatgaacatccaggccccgggacctcagactgctcattggctgcctgaaaacccgcccccaagcctgagagaaaagaactctggttgctgtagttccccaacgaacaccgcagaaag

SEQ ID NO: 21 - DNA, 618 bp, Homo sapiens
  misc_feature (1)..(618): cg20655070 DNA sequence after bisulfite conversion
  misc_feature (204)..(324): DNA sequence amplified by qMSP
ttttttgcggtgttcgttggggaattatagtaattagagttttttttttttaggtttgggggcgggtttttaggtagttaatgagtagtttgaggtttcggggtttggatgtttatggtgtttaggagagggagagaaggttgtgattgggtggttttggcgtaggtgttgaggttttcgaggtgtgtagaaggtgggtgcggcgtcgtttcgggagagagtgtcggttgtcgtttatagttttagagcgggtagtagacgtcgaggtttgaagaggttttagtacgcggatattattggtcgttgtgtttggttcggtttgcgggaagtttcgtttagtgggagagataggtaagatttcgttgcggatttatgtatgtttattaatggaggcggtatggaacgcggtgggaattcgaggagattttggtaggaagattatttcggaagattaggagttgtttgagttgagatttgaagacggaaggatgttttgggggtcgggggcggtgtggtggaatggtttgcgtaaaggtagagagaaatagtttgttgtgtgaatatggattgtaggcgatttggtgttggtgattatattgaaaattttgtatgaatgag

SEQ ID NO: 22 - DNA, 1886 bp, Homo sapiens
  misc_feature (1)..(1886): TAC1 original DNA sequence, chr797361133- 97363018
cgtcagatctgcagacggaagcaggccgctccggattggatggcgagacctcgattttcctaaaattgcgtcatttagaacccaattgggtccagatgttatgggcatcgacgagttaccgtctcggaaactctcaatcacgcaagcgaaaggagaggaggcggctaattaaatattgagcagaaagtcgcgtggggagaatgtcacgtgggtctggaggctcaaggaggctgggataaataccgcaaggcactgagcaggcgaaagagcgcgctcggacctccttcccggcggcagctaccgagagtgcggagcgaccagcgtgcgctcggaggaaccagagaaactcagcaccccgcgggactgtccgtcgcagtaagtgcccgcgcggtgctggccgcggctgcccgggtcaccccgccccgcatctgtccgaggtggccgcgctgggggcgccgctgcggcgagggacagtggggagactggcttcccaaacgccaacgcccctctttgtcttccacctgcagagtttcctggtttgaaggtgtgggttggtgggttagggggctgggggagttgggattcagggagaagagggttggagaatctttgggacgcgattctctcgcctaaccggtacaggtgagacttcagtccttatgtttttgatcttggttcatccgttgtggggcagaaaattctgttgctttaactcttggataaccacccctaatagatacattatttctctctttggtgtcttctcctcctaccccttcccagaaatccaacatgaaaatcctcgtggccttggcagtcttttttcttgtctccactcagctgtttgcagaagaaataggagccaatgatgatctgaattactggtccgactggtacgacagcgaccagatcaaggtgaggccccttcccaggacggcccgcacccttcttcctgggctcgggagctgtcaccttcccacgcaacagcaccctagttaacgtggcacgcaccgccaccacggaaagaggcagcggttgcgtgcgagaggatggaaaggggcactatttcccgggttccccacgggattttgtgcccacgattcaagtttcttcccgagggctgcaccgtccggcccaggaactccctgcagtagggatgccctcccggatgagcccgagatcctcacaaggcgggaaatctcgtaagatttcatcccccaggacctgggatttcgggggctccggtccagctgcactgccctagcgtctgggcacgggcagaggagggcgcctgggtcgcgggcagcagcgggcacacgcgcgagctgcagggggattggagggccggaggctggagggaagttaatttcctgccttcacgtttgttttgcggacctgaggatgtagcttgaagtctccctttagaaactcgtaggctacaggcgctggggagcgcgcctcggaaaaacggtgaaagtcgtctgtaaagcgttgtcacccttccccaccggagccccagccccagatcccacacacctgtgtgcatttagggcggtgaggactctacaaagccggcaccgagacaagacaaagtcaggtggcggcgcagcagatgagggaaggtccccgggctgggaggaaagagcggaaagcgctcgaagcttgaagacgccgagccagctgctaactcgccctggcaggcttgcggggggcgggacgcgagttcggcggggaggggcgcagagaggcagcgcctcgggagggaccccgctcagcccgagcgtggggaaggccgcctccccacgcctcggggccggagtacagcggggggaagggctcgggttgctgggtgcctcgctctggttgccttacacgccctttgtccg

SEQ ID NO: 23 - DNA, 1886 bp, Homo sapiens
  misc_feature (1)..(1886): TAC1 DNA sequence after bisulfite conversion
  misc_feature (103)..(208): DNA sequence amplified by qMSP
cgttagatttgtagacggaagtaggtcgtttcggattggatggcgagatttcgatttttttaaaattgcgttatttagaatttaattgggtttagatgttatgggtatcgacgagttatcgtttcggaaatttttaattacgtaagcgaaaggagaggaggcggttaattaaatattgagtagaaagtcgcgtggggagaatgttacgtgggtttggaggtttaaggaggttgggataaatatcgtaaggtattgagtaggcgaaagagcgcgttcggatttttttttcggcggtagttatcgagagtgcggagcgattagcgtgcgttcggaggaattagagaaatttagtatttcgcgggattgttcgtcgtagtaagtgttcgcgcggtgttggtcgcggttgttcgggttatttcgtttcgtatttgttcgaggtggtcgcgttgggggcgtcgttgcggcgagggatagtggggagattggttttttaaacgttaacgtttttttttgttttttatttgtagagttttttggtttgaaggtgtgggttggtgggttagggggttgggggagttgggatttagggagaagagggttggagaatttttgggacgcgattttttcgtttaatcggtataggtgagattttagtttttatgtttttgattttggtttattcgttgtggggtagaaaattttgttgttttaatttttggataattatttttaatagatatattatttttttttttggtgttttttttttttatttttttttagaaatttaatatgaaaattttcgtggttttggtagtttttttttttgtttttatttagttgtttgtagaagaaataggagttaatgatgatttgaattattggttcgattggtacgatagcgattagattaaggtgaggtttttttttaggacggttcgtatttttttttttgggttcgggagttgttatttttttacgtaatagtattttagttaacgtggtacgtatcgttattacggaaagaggtagcggttgcgtgcgagaggatggaaaggggtattatttttcgggttttttacgggattttgtgtttacgatttaagttttttttcgagggttgtatcgttcggtttaggaattttttgtagtagggatgttttttcggatgagttcgagatttttataaggcgggaaatttcgtaagattttattttttaggatttgggatttcgggggtttcggtttagttgtattgttttagcgtttgggtacgggtagaggagggcgtttgggtcgcgggtagtagcgggtatacgcgcgagttgtagggggattggagggtcggaggttggagggaagttaattttttgtttttacgtttgttttgcggatttgaggatgtagtttgaagtttttttttagaaattcgtaggttataggcgttggggagcgcgtttcggaaaaacggtgaaagtcgtttgtaaagcgttgttatttttttttatcggagttttagttttagattttatatatttgtgtgtatttagggcggtgaggattttataaagtcggtatcgagataagataaagttaggtggcggcgtagtagatgagggaaggttttcgggttgggaggaaagagcggaaagcgttcgaagtttgaagacgtcgagttagttgttaattcgttttggtaggtttgcggggggcgggacgcgagttcggcggggaggggcgtagagaggtagcgtttcgggagggatttcgtttagttcgagcgtggggaaggtcgtttttttacgtttcggggtcggagtatagcggggggaagggttcgggttgttgggtgtttcgttttggttgttttatacgttttttgttcg

SEQ ID NO: 24 - DNA, 1769 bp, Homo sapiens
  misc_feature (1)..(1769): SLC35F1 original DNA sequence, chr6118228043- 118229811
cgtttgggtttctaatccggggccggcacctgtctcggcgtcctgccgctgtcccaggccggtggtgctggattccgcggcgcttgggcagaaacgaggaatccccgccaactccacagggaggatctattcccagcccccttatccccctaaaaaacacccaaaccaaaacaatcaccacaacctagaccgtgggcctctgcggacggggcagtagagggtcgggggcaacaaggcacctcggcagcgccctctggcgagaggagagcgcgcaggtcccccgcccgcgccccgctctgccgccgccgccgccgccgccgccgccgccgccggcgcccccgcccggctctccgggttcaccctcggtctctcactccgtgagacacagacggtagctttccgaccgagcggggcacaggctgaggcggcgccagcacaactgccgccgcgcgccccgaggagacccgggcacgaggaaacaaggagaaatcaggactccttccccggaaccgaccggcagctccgcgcccccgcgcgacacccttcgcagccacttcgcggggcgggccaggacttggggacgcggctcgggaagagccggggcgggcggcggcggcggcggcacgggcgcgagggtgcgcgcactgggactggagaggagtgagtggcgggctgggcggcggcggccgtagccgcgggtgcctccccgcctcaccgcttcgcaggcagcaccgcctcccgggccgcggccgcccggccgggtcctctgcgcgttcccgggcccggaaccggcacacgatgcacccggctgcgttctgatcgccgccgcgcctcagcctctgccgcgatgatcccccctgagcagccgcagcagcagctgcagccgccgtcgccagccccgccgaaccatgtggtgaccaccatcgagaacctgccggccgagggcagcggcggcggcgggagcctgtccgcctcctcccgggctggcgtgcgccagaggatccgcaaagtgctgaacaggtgagcggcggcgccgggcgagggcgcggggggcgggggctgcgggcgcccggctccgggtcccctccgtccctggggcggcccacgggtcctttgggacggggttgcggccggaggagactgaggagatcgggcgacgacgcgctccgcgggcgcagagaggccgcggcagctttgtcccggcgcagggagccctggagtggtggagctgcagctggtccgaggcgggagttcggggagaggcggggtttttcgtacctagccaccctgtatgtgtgtccgcactcacaccctccatacgcgtgccgcctcacttccctttccctctcgcaccccggctttgcgatcgggggtcggggggacgcggggacccggccagggctgaggagggaggcgcggggctgcagagatggcgtggaagtcctgggcctcgtgctgtcctccgccccgccccgcccgggggcttcgaccccttgccgggggaatggggtcgcagcgacagcctagcaggcggagggaacccacttctccagcctgacgctccagtcgcctctcggactcttcctggctgggagcagcgaagagagggctagaggcctggaacagacccagcaaagtgcaaaacattgcgggcagggtgtgagccggcggcgcccagttattccgaagcgcccatgccggagggggcgggtgccgatcgccggcgcctccagtgctggaatctgcgtagtggattcaggacgttgacg

SEQ ID NO: 25 - DNA, 1769 bp, Homo sapiens
  misc_feature (1)..(1769): SLC35F1 DNA sequence after bisulfite conversion
  misc_feature (867)..(1014): DNA sequence amplified by qMSP
cgtttgggtttttaattcggggtcggtatttgtttcggcgttttgtcgttgttttaggtcggtggtgttggatttcgcggcgtttgggtagaaacgaggaattttcgttaattttatagggaggatttatttttagtttttttatttttttaaaaaatatttaaattaaaataattattataatttagatcgtgggtttttgcggacggggtagtagagggtcgggggtaataaggtatttcggtagcgttttttggcgagaggagagcgcgtaggtttttcgttcgcgtttcgttttgtcgtcgtcgtcgtcgtcgtcgtcgtcgtcgtcggcgttttcgttcggtttttcgggtttattttcggttttttatttcgtgagatatagacggtagtttttcgatcgagcggggtataggttgaggcggcgttagtataattgtcgtcgcgcgtttcgaggagattcgggtacgaggaaataaggagaaattaggatttttttttcggaatcgatcggtagtttcgcgttttcgcgcgatatttttcgtagttatttcgcggggcgggttaggatttggggacgcggttcgggaagagtcggggcgggcggcggcggcggcggtacgggcgcgagggtgcgcgtattgggattggagaggagtgagtggcgggttgggcggcggcggtcgtagtcgcgggtgttttttcgttttatcgtttcgtaggtagtatcgtttttcgggtcgcggtcgttcggtcgggttttttgcgcgttttcgggttcggaatcggtatacgatgtattcggttgcgttttgatcgtcgtcgcgttttagtttttgtcgcgatgattttttttgagtagtcgtagtagtagttgtagtcgtcgtcgttagtttcgtcgaattatgtggtgattattatcgagaatttgtcggtcgagggtagcggcggcggcgggagtttgttcgttttttttcgggttggcgtgcgttagaggattcgtaaagtgttgaataggtgagcggcggcgtcgggcgagggcgcggggggcgggggttgcgggcgttcggtttcgggtttttttcgtttttggggcggtttacgggttttttgggacggggttgcggtcggaggagattgaggagatcgggcgacgacgcgtttcgcgggcgtagagaggtcgcggtagttttgtttcggcgtagggagttttggagtggtggagttgtagttggttcgaggcgggagttcggggagaggcggggtttttcgtatttagttattttgtatgtgtgttcgtatttatattttttatacgcgtgtcgttttatttttttttttttttcgtatttcggttttgcgatcgggggtcggggggacgcggggattcggttagggttgaggagggaggcgcggggttgtagagatggcgtggaagttttgggtttcgtgttgtttttcgtttcgtttcgttcgggggtttcgattttttgtcgggggaatggggtcgtagcgatagtttagtaggcggagggaatttatttttttagtttgacgttttagtcgtttttcggattttttttggttgggagtagcgaagagagggttagaggtttggaatagatttagtaaagtgtaaaatattgcgggtagggtgtgagtcggcggcgtttagttatttcgaagcgtttatgtcggagggggcgggtgtcgatcgtcggcgtttttagtgttggaatttgcgtagtggatttaggacgttgacg